(19) United States
(12) Patent Application Publication
(10) Pub. No.: US 2020/0140835 A1
(43) Pub. Date: May 7, 2020

(54) Engineered CRISPR-Cas9 Nucleases

(71) Applicants: The General Hospital Corporation, Boston, MA (US); The Regents of the University of California, Oakland, CA (US)

(72) Inventors: J. Keith Joung, Winchester, MA (US); Benjamin Kleinstiver, Medford, MA (US); Janice Sha Chen, Berkeley, CA (US); Jennifer Doudna, Berkeley, CA (US); Yavuz Selim Dagdas, Albany, CA (US); Ahmet Yildiz, El Cerrito, CA (US)

(21) Appl. No.: 16/620,367

(22) PCT Filed: Jun. 6, 2018
(86) PCT No.: PCT/US2018/036293

§ 371 Date: Dec. 6, 2019

Related U.S. Application Data
(60) Provisional application No. 62/515,938, filed on Jun. 6, 2017.

(51) Int. Cl. C12N 9/22; C12N 15/85; C12N 15/90; C12N 9/02; C07K 14/47

(52) U.S. Cl. CPC C12N 9/22; C12N 15/85; C12N 15/907; C12N 9/0071; C12N 2310/20; C07K 2319/80; C07K 2319/71; C12N 2800/80; C07K 14/4705

(57) ABSTRACT

Engineered CRISPR-Cas9 nucleases with improved specificity and their use in genomic engineering, epigenomic engineering, genome targeting, and genome editing.

### CLAIM OF PRIORITY

This application claims the benefit of U.S. Provisional Patent Application Ser. No. 62/515,938, filed on Jun. 6, 2017. The entire contents of the foregoing are hereby incorporated by reference.

### FEDERALLY SPONSORED RESEARCH OR DEVELOPMENT

This invention was made with Government support under Grant Nos. GM118158 GM105378 and GM088040 awarded by the National Institutes of Health and Grant Nos. 0187644 and MCB-1244557 awarded by the National Science Foundation. The Government has certain rights in the invention.

### TECHNICAL FIELD

Engineered CRISPR-Cas9 nucleases with improved specificity and their use in genomic engineering, epigenomic engineering, genome targeting, and genome editing.

### BACKGROUND

The RNA-guided CRISPR-Cas9 nuclease from Streptococcus pyogenes (SpCas9) has been widely repurposed for genome editing¹⁻³. High-fidelity (SpCas9-HF1; see WO 2017/040348) and enhanced specificity (eSpCas9(1.1); WO 2016/205613) variants have substantially reduced off-target cleavage in human cells, but the mechanism of target discrimination and the potential to further improve fidelity were unknown⁴⁻⁸.

### SUMMARY

Using single-molecule Förster resonance energy transfer (smFRET) experiments, we show that contrary to initial predictions, both SpCas9-HF1 and eSpCas9(1.1) are trapped in an inactive state⁹ when complexed with a guide RNA and bound to mismatched targets. Mismatches sensed by one domain of the enzyme control the conformational activation of the Cas9 HNH nuclease, thereby regulating overall catalytic competence. Exploiting this observation, residues involved in mismatch sensing were identified and this information was utilized to create new hyper-accurate Cas9 variants that retain robust on-target activity in human cells and methods of using them described herein.

Thus, provided herein are isolated Streptococcus pyogenes Cas9 (SpCas9) proteins, with mutations at one, two, three, four, five, six, seven, or more of the following positions: F491, M495, T496, N497, G582, V583, E584, D585, N588, T657, G658, W659, R661, N692, M694, Q695, H698, K918, V922, and/or R925, and optionally at Q926, preferably comprising a sequence that is at least 80% identical to the amino acid sequence of SEQ ID NO:1 with mutations at one, two, three, four, five, six, seven, or more of the following positions: F491, M495, T496, N497, G582, V583, E584, D585, N588, T657, G658, W659, R661, N692, M694, Q695, H698, K918, V922, and/or R925, and optionally at Q926, and optionally one or more of a nuclear localization sequence, cell penetrating peptide sequence, and/or affinity tag.

In some embodiments, the proteins comprise mutations at one, two, three, or all four of the following: N692, M694, Q695, and H698; G582, V583, E584, D585, and N588; T657, G658, W659, and R661; F491, M495, T496, and N497; or K918, V922, R925, and Q926.

In some embodiments, the proteins comprise one, two, three, four, or all of the following mutations: N692A, M694A, Q695A, and H698A; G582A, V583A, E584A, D585A, and N588A; T657A, G658A, W659A, and R661A; F491A, M495A, T496A, and N497A; or K918A, V922A, R925A, and Q926A.

In some embodiments, the proteins comprise mutations: N692A/M694A/Q695A/H698A.

In some embodiments, the proteins comprise mutations: N692A/M694A/Q695A/H698A/Q926A; N692A/M694A/Q695A/Q926A; N692A/M694A/H698A/Q926A; N692A/Q695A/H698A/Q926A; M694A/Q695A/H698A/Q926A; N692A/Q695A/H698A; N692A/M694A/Q695A; N692A/H698A/Q926A; N692A/M694A/Q926A; N692A/M694A/H698A; M694A/Q695A/H698A; M694A/Q695A/Q926A; Q695A/H698A/Q926A; G582A/V583A/E584A/D585A/N588A/Q926A; G582A/V583A/E584A/D585A/N588A; T657A/G658A/W659A/R661A/Q926A; T657A/G658A/W659A/R661A; F491A/M495A/T496A/N497A/Q926A; F491A/M495A/T496A/N497A; K918A/V922A/R925A/Q926A; or 918A/V922A/R925A.

In some embodiments, the proteins also comprise one or more of the following mutations: D1135E; D1135V; G1218R; R1335Q; R1335E; T1337R; D1135V/R1335Q/T1337R (VQR variant); D1135E/R1335Q/T1337R (EQR variant); D1135V/G1218R/R1335Q/T1337R (VRQR variant); or D1135V/G1218R/R1335E/T1337R (VRER variant).

In some embodiments, the proteins also comprise one or more mutations that decrease nuclease activity selected from the group consisting of mutations at D10, E762, D839, H983, or D986; and at H840 or N863. In some embodiments, the mutations that decrease nuclease activity are: (i) D10A or D10N, and (ii) H840A, H840N, or H840Y.

Also provided herein are fusion proteins comprising the Vas9 variant proteins described herein, preferably comprising one or more mutations that decrease nuclease activity, fused to a heterologous functional domain, with an optional intervening linker, wherein the linker does not interfere with activity of the fusion protein.

In some embodiments, the heterologous functional domain is a transcriptional activation domain, e.g., from VP64 or NF-κB p65.

In some embodiments, the heterologous functional domain is a transcriptional repression domain, e.g., a Krueppel-associated box (KRAB) domain, ERF repressor domain (ERD), or mSin3A interaction domain (SID), or a transcriptional silencer, e.g., Protein 1 (HP1), preferably HP1α or HP1β.

In some embodiments, the heterologous functional domain is an enzyme that modifies the methylation state of DNA, e.g., a DNA methyltransferase (DNMT) or a TET protein (e.g., TET1).

In some embodiments, the heterologous functional domain is an enzyme that modifies a histone subunit, e.g., a histone acetyltransferase (HAT), histone deacetylase (HDAC), histone methyltransferase (HMT), or histone demethylase.

In some embodiments, the heterologous functional domain is a biological tether, e.g., MS2, Csy4 or lambda N protein.

In some embodiments, the heterologous functional domain is FokI.

In some embodiments, the heterologous functional domain comprises a deaminase enzyme, e.g., a cytidine deaminase, and optionally a uracil glycosylase inhibitor (UGI) domain.

Also provided herein are isolated nucleic acids encoding the Cas9 variant proteins and fusion proteins described herein, as well as vectors comprising the isolated nucleic acid, optionally operably linked to one or more regulatory domains for expressing the protein or the fusion protein.

Further provided herein are host cells, preferably mammalian host cells, comprising the nucleic acids described herein, and optionally expressing a Cas9 variant protein or fusion protein as described herein.

A method of altering the genome of a cell, the method comprising expressing in the cell or contacting the cell with a Cas9 variant protein or fusion protein as described herein, and a guide RNA having a region complementary to a selected portion of the genome of the cell.

In some embodiments, the isolated protein or fusion protein comprises one or more of a nuclear localization sequence, cell penetrating peptide sequence, and/or affinity tag.

In some embodiments, the cell is a stem cell, e.g., an embryonic stem cell, mesenchymal stem cell, or induced pluripotent stem cell; is in a living animal; or is in an embryo.

A method of altering a double stranded DNA (dsDNA) molecule, the method comprising contacting the dsDNA molecule with a Cas9 variant protein or fusion protein as described herein, and a guide RNA having a region complementary to a selected portion of the dsDNA molecule.

In some embodiments, the dsDNA molecule is in vitro.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-F| High-fidelity Cas9 variants enhance cleavage specificity through HNH conformational control. A, Cartoon illustrating locations of amino acid alterations present in existing high-fidelity SpCas9 variants mapped onto the dsDNA-bound SpCas9 crystal structure (5F9R), with the HNH domain omitted for clarity. B, Dissociation constants comparing WT SpCas9, SpCas9-HF1 and eSpCas9(1.1) with perfect and a 20-16 bp mismatched target. C, Cartoon of DNA-immobilized SpCas9 complexes for smFRET experiments with DNA target numbering scheme. D-F, smFRET histograms measuring HNH conformational activation with D, WT SpCas9_{HNH}, E, SpCas9-HF1_{HNH} and F, eSpCas9(1.1)_{HNH} bound to perfect and PAM-distal mismatched targets. Black curves represent a fit to multiple Gaussian peaks.

FIGS. 2A-H| Helical-III domain is an activator of the HNH nuclease domain. A, Schematic of SpCas9_{Helical-III} with FRET dyes at positions S701C and S960C, with HNH domain omitted for clarity. Inactive to active structures represent Helical-III in the sgRNA-bound (PDB ID: 4ZT0) to dsDNA-bound (PDB ID: 5F9R) forms, respectively. B, smFRET histograms measuring HNH conformational activation with WT SpCas9_{Helical-III} and C, SpCas9-HF1_{Helical-III} bound to perfect and PAM-distal mismatched targets. D, Domain organization of SpCas9ΔHelical-III (ΔF498-Q712 with GGS linker). E, Perfect target DNA cleavage assay using SpCas9ΔHelical-III with increasing concentrations of Helical-III domain supplied in trans, resolved by denaturing PAGE. F, Perfectly matched on-target DNA binding assay in the presence or absence of Helical-III domain. G, Cleavage rate constants using SpCas9ΔHelical-III and H, (Ratio)_{A} data using SpCas9ΔHelical-III_{HNH} with Helical-III supplied in trans on a perfect target and 1-4 bp mismatched substrate. For panels B and C, black curves represent a fit to multiple Gaussian peaks.

FIGS. 3A-E| Targeted mutagenesis within the Helical-III domain reveals a SpCas9 variant with hyper-accurate behavior in human cells. A, Zoomed image of Helical-III domain and Linker 2 (L2) with Cluster variants indicated. B, WT-normalized activity of SpCas9-HF1, eSpCas9(1.1) and Cluster variants with or without Q926A, using 12 different sgRNAs targeted to EGFP. C, WT-normalized endogenous gene disruption activity measured by T7E1 assay across 24 sites for SpCas9-HF1, eSpCas9(1.1) and Cluster 1 A926Q. D, Activities of WT SpCas9, SpCas9-HF1, eSpCas9(1.1) and Cluster variants when programmed with singly mismatched sgRNAs against FANCF site 1. E, Activities of WT SpCas9, SpCas9-HF1, eSpCas9(1.1) and Cluster 1 A926Q when programmed with singly mismatched sgRNAs against FANCF site 4 and FANCF site 6. For panels B and C, error bars represent median and interquartile ranges, and n 3; the interval with >70% of wild-type activity is highlighted in light grey.

FIGS. 4A-B| Real-time kinetics of HNH docking upon DNA binding using high-fidelity Cas9 complexes and model for alpha-helical lobe sensing. A, smFRET histograms measuring HNH, Helical-II and Helical-III conformational states for HypaCas9; black curves represent a fit to multiple Gaussian peaks. B, Model for alpha-helical lobe sensing and regulation of the RNA/DNA heteroduplex for HNH activation and cleavage.

FIGS. 5A-E| Dually-labeled SpCas9 is fully functional for DNA cleavage. A, Sodium dodecyl sulphate-polyacrylamide gel electrophoresis (SDS-PAGE) analysis of unlabeled Cas9 variants. B, SDS-PAGE analysis of Cy3/Cy5-labeled Cas9 variants. The gel was scanned for Cy3/Cy5 fluorescence (middle, bottom) before staining with Coomassie blue (top). C-E, DNA cleavage time courses of Cas9 FRET constructs and their dually-labeled counterparts for C, WT SpCas9, D, SpCasy-HF1, and E, eSpCas9(1.1).

FIGS. 6A-D| HNH domain in eSpCas9 variants still populate the docked state in the presence of PAM-distal mismatches. A, Dissociation constants comparing WT SpCas9, SpCas9-HF and eSpCas9(1.1) variants with perfect and PAM-distal mismatched targets. B-C, smFRET histograms for B, SpCas9-K855A and C, SpCas9-N497A/R661A/Q695A. D, Quantification of DNA cleavage time courses comparing WT SpCas9, SpCas9-HF and eSpCas9(1.1) variants with perfect and PAM-distal mismatched targets.

FIGS. 7A-B| The HNH nuclease, Helical-II and Helical-III domains undergo substantial conformational changes upon binding to the dsDNA target. A, Schematic of SpCas9 domain structure with color coding for separate domains. B, Vector map of global SpCas9 conformational changes from the sgRNA- (PDB ID: 4ZT0)¹⁴ to dsDNA-bound structures (PDB ID: 5F9R)¹⁵; domains colored as in panel A.

FIGS. 8A-D| Helical-II domain regulates HNH activation by steric occlusion. A, Schematic of SpCas9_{Helical-II} with FRET dyes at positions E60C and D273C, with HNH domain omitted for clarity. Inactive to active structures represent Helical-II in the sgRNA-bound (PDB ID: 4ZT0) to dsDNA-bound (PDB ID: 5F9R) forms, respectively. B, (Ratio)_{A} data with SpCas9_{Helical-II} and SpCas9H showing reciprocal FRET states with the indicated substrates. C-D, smFRET histograms measuring HNH conformational activation with C, WT SpCas9_{Helical-II} and D, SpCas9-HF1_{Helical-II} bound to perfect and PAM-distal mismatched targets.

FIGS. 9A-D| Mutation clusters in Helical-III domain along the RNA/DNA heteroduplex demonstrate localized sensitivity to mismatches along the target sequence. A-B, Target DNA binding assay A, resolved by native polyacrylamide gel electrophoresis (PAGE) mobility shift assays and B, quantification with WT-normalized dissociation constants. C-D, Quantified DNA cleavage rates (detection limit for k_{cleave} set at 10 min′) displayed as a C, heatmap and D, bar graph with mean and SD, where n=3.

FIGS. 10A-C| On-target activities of altered specificity variants using a human cell EGFP disruption assay. A, Summary of EGFP disruption activities for SpCas9-HF1, eSpCas9(1.1), eSpCas9(1.1)-HF1 and Cluster variants±Q926A with mean and s.e.m., where n≥3. B, Summary of EGFP disruption activities for the series of Cluster 1 variants with each substituted residue restored to the canonical amino acid; mean and s.e.m. where n≥3; WT, Cluster 1, and Cluster 1 A926Q data from panel A is re-plotted for comparison. C, WT-normalized plot of data in panel B; error bars represent median and interquartile range; the interval with >70% of wild-type activity is highlighted in light grey.

FIGS. 11A-E| Activities and specificities of high-fidelity SpCas9 variants targeted to endogenous human cell sites. A, On-target activities of WT SpCas9, SpCas9-HF1, Cluster 1 and Cluster 2 variants across 24 endogenous human genes, assessed by T7E1 assay. Mean and s.e.m. shown; n≥3. B, WT-normalized endogenous gene disruption data from panel A, for Cluster 1 and 2 variants. Error bars represent median and interquartile ranges with the >70% interval of wild-type activity highlighted in light grey; Cluster 1 A926Q data from FIG. 3B is re-plotted for comparison. C-E, Summary of single mismatch tolerance of WT SpCas9, SpCas9-HF1, eSpCas9(1.1), and Cluster 1 and Cluster 2 variants on C, FANCF site 1 D, FANCF sites 4 and 6, and E, FANCF site 2. Percent modification assessed by T7E1 assay; mean and s.e.m. shown; n≥3.

FIG. 12| All enhanced specificity, high-fidelity and cluster SpCas9 variants tested in this study; plasmids deposited on Addgene are indicated. The HNH, Helical-II or Helical-III subscript designation with an enhanced specificity, high-fidelity or cluster SpCas9 variant denotes combination of residue substitutions with indicated FRET construct.

### DETAILED DESCRIPTION

A limitation of the CRISPR-Cas9 nucleases is their potential to induce undesired “off-target” mutations at imperfectly matched target sites (see, for example, Tsai et al., Nat Biotechnol. 2015), in some cases with frequencies rivaling those observed at the intended on-target site (Fu et al., Nat Biotechnol. 2013). Existing strategies for generating high-fidelity SpCas9 complexes include protein engineering and guide RNA modifications, but how these protein variants achieve a greater differential between on- and off-targeting remains unclear. Using biochemical and single-molecule Førster resonance energy transfer experiments, the present inventors observed that high-fidelity SpCas9 complexes do not affect target binding affinity, but instead structurally “trap” the HNH nuclease in an inactive conformation when bound to mismatched targets. Described herein are key roles of Helical-II and Helical-III protein domains in target recognition and HNH nuclease regulation, and residue clusters that allosterically control SpCas9 conformational activation to tune in vitro and in vivo specificity. As described herein, Cas9 proteins were engineered based on these findings to show increased specificities.

All of the variants described herein can be rapidly incorporated into existing and widely used vectors, e.g., by simple site-directed mutagenesis, and because they require only a small number of mutations, the variants should also work with other previously described improvements to the SpCas9 platform (e.g., truncated sgRNAs (Tsai et al., Nat Biotechnol 33, 187-197 (2015); Fu et al., Nat Biotechnol 32, 279-284 (2014)), nickase mutations (Mali et al., Nat Biotechnol 31, 833-838 (2013); Ran et al., Cell 154, 1380-1389 (2013)), FokI-dCas9 fusions (Guilinger et al., Nat Biotechnol 32, 577-582 (2014); Tsai et al., Nat Biotechnol 32, 569-576 (2014); WO2014144288); and engineered CRISPR-Cas9 nucleases with altered PAM specificities (Kleinstiver et al., Nature. 2015 Jul. 23; 523(7561):481-5).

Thus, provided herein are Cas9 variants, including SpCas9 variants. The SpCas9 wild type sequence is as follows:

```
(SEQ ID NO: 1)
                10 20 30 40
MDKKYSIGLD IGTNSVGWAV ITDEYKVPSK KFKVLGNTDR
                50 60 70 80
HSIKKNLIGA LLFDSGETAE ATRLKRTARR RYTRRKNRIC
                90 100 110 120
YLQEIFSNEM AKVDDSFFHR LEESFLVEED KKHERHPIFG
              130 140 150 160
NIVDEVAYHE KYPTIYHLRK KLVDSTDKAD LRLIYLALAH
              170 180 190 200
MIKFRGHFLI EGDLNPDNSD VDKLFIQLVQ TYNQLFEENP
              210 220 230 240
INASGVDAKA ILSARLSKSR RLENLIAQLP GEKKNGLFGN
              250 260 270 280
LIALSLGLTP NFKSNFDLAE DAKLQLSKDT YDDDLDNLLA
              290 300 310 320
QIGDQYADLF LAAKNLSDAI LLSDILRVNT EITKAPLSAS
              330 340 350 360
MIKRYDEHHQ DLTLLKALVR QQLPEKYKEI FFDQSKNGYA
              370 380 390 400
GYIDGGASQE EFYKFIKPIL EKMDGTEELL VKLNREDLLR
              410 420 430 440
KQRTFDNGSI PHQIHLGELH AILRRQEDFY PFLKDNREKI
              450 460 470 480
EKILTFRIPY YVGPLARGNS RFAWMTRKSE ETITPWNFEE
              490 500 510 520
VVDKGASAQS FIERMINFDK NLPNEKVLPK HSLLYEYFTV
              530 540 550 560
YNELTKVKYV TEGMRKPAFL SGEQKKAIVD LLFKTNRKVT
              570 580 590 600
VKQLKEDYFK KIECFDSVEI SGVEDRFNAS LGTYHDLLKI
              610 620 630 640
IKDKDFLDNE ENEDILEDIV LTLTLFEDRE MIEERLKTYA
              650 660 670 680
HLFDDKVMKQ LKRRRYTGWG RLSRKLINGI RDKQSGKTIL
              690 700 710 720
DFLKSDGFAN RNFMQLIHDD SLTFKEDIQK AQVSGQGDSL
              730 740 750 760
HEHIANLAGS PAIKKGILQT VKVVDELVKV MGRHKPENIV
              770 780 790 800
IEMARENQTT QKGQKNSRER MKRIEEGIKE LGSQILKEHP
              810 820 830 840
VENTQLQNEK LYLYYLQNGR DMYVDQELDI NRLSDYDVDH
              850 860 870 880
IVPQSFLKDD SIDNKVLTRS DKNRGKSDNV PSEEVVKKMK
              890 900 910 920
NYWRQLLNAK LITQRKFDNL TKAERGGLSE LDKAGFIKRQ
              930 940 950 960
LVETRQITKH VAQILDSRMN TKYDENDKLI REVKVITLKS
              970 980 990 1000
KLVSDFRKDF QFYKVREINN YHHAHDAYLN AVVGTALIKK
            1010 1020 1030 1040
YPKLESEFVY GDYKVYDVRK MIAKSEQEIG KATAKYFFYS
            1050 1060 1070 1080
NIMNFFKTEI TLANGEIRKR PLIETNGETG EIVWDKGRDF
            1090 1100 1110 1120
ATVRKVLSMP QVNIVKKTEV QTGGFSKESI LPKRNSDKLI
            1130 1140 1150 1160
ARKKDWDPKK YGGFDSPTVA YSVLVVAKVE KGKSKKLKSV
            1170 1180 1190 1200
KELLGITIME RSSFEKNPID FLEAKGYKEV KKDLIIKLPK
            1210 1220 1230 1240
YSLFELENGR KRMLASAGEL QKGNELALPS KYVNFLYLAS
            1250 1260 1270 1280
HYEKLKGSPE DNEQKQLFVE QHKHYLDEII EQISEFSKRV
            1290 1300 1310 1320
ILADANLDKV LSAYNKHRDK PIREQAENII HLFTLTNLGA
            1330 1340 1350 1360
PAAFKYFDTT IDRKRYTSTK EVLDATLIHQ SITGLYETRI
DLSQLGGD
```

The SpCas9 variants described herein can include the amino acid sequence of SEQ ID NO:1, with mutations (i.e., replacement of the native amino acid with a different amino acid, e.g., alanine, glycine, or serine), at one or more of the following positions: F491, M495, T496, N497, G582, V583, E584, D585, N588, T657, G658, W659, R661, N692, M694, Q695, H698, K918, V922, R925, and Q926 (or at positions analogous thereto); where Q926 is mutated, at least one of the other residues is also mutated. In some embodiments, the SpCas9 variants are at least 80%, e.g., at least 85%, 90%, or 95% identical to the amino acid sequence of SEQ ID NO:1, e.g., have differences at up to 5%, 10%, 15%, or 20% of the residues of SEQ ID NO:1 replaced, e.g., with conservative mutations, in addition to the mutations described herein. In preferred embodiments, the variant retains desired activity of the parent, e.g., the nuclease activity (except where the parent is a nickase or a dead Cas9), and/or the ability to interact with a guide RNA and target DNA).

To determine the percent identity of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The nucleotides at corresponding amino acid positions or nucleotide positions are then compared. When a position in the first sequence is occupied by the same nucleotide as the corresponding position in the second sequence, then the molecules are identical at that position (as used herein nucleic acid “identity” is equivalent to nucleic acid “homology”). The percent identity between the two sequences is a function of the number of identical positions shared by the sequences, taking into account the number of gaps, and the length of each gap, which need to be introduced for optimal alignment of the two sequences. Percent identity between two polypeptides or nucleic acid sequences is determined in various ways that are within the skill in the art, for instance, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M. O., Ed, pp 353-358; BLAST program (Basic Local Alignment Search Tool; (Altschul, S. F., W. Gish, et al. (1990) J Mol Biol 215: 403-10), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. In general, for proteins or nucleic acids, the length of comparison can be any length, up to and including full length (e.g., 5%, 10%, 20%, 30%, 40%, 50%, 60%, 70%, 80%, 90%, 95%, or 100%). For purposes of the present compositions and methods, at least 80% of the full length of the sequence is aligned.

For purposes of the present invention, the comparison of sequences and determination of percent identity between two sequences can be accomplished using a Blossum 62 scoring matrix with a gap penalty of 12, a gap extend penalty of 4, and a frameshift gap penalty of 5.

Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

In some embodiments, the SpCas9 variants include mutations at one of the following clusters of residues:
Residue Cluster 1: N692, M694, Q695, H698
Residue Cluster 2: G582, V583, E584, D585, N588
Residue Cluster 3: T657, G658, W659, R661
Residue Cluster 4: F491, M495, T496, N497
Residue Cluster 5: K918, V922, R925, Q926

In some embodiments, the mutants can have alanine in place of the wild type amino acid. In some embodiments, the mutants can have any amino acid other than arginine, lysine, asparagine, or glutamine (or the native amino acid). In some embodiments, the SpCas9 variants include one, two, three, four, five or more of the following mutations: F491A, M495A, T496A, N497A, G582A, V583A, E584A, D585A, N588A, T657A, G658A, W659A, R661A, N692A, M694A, Q695A, H698A, K918A, V922A, R925A, and/or Q926A.

In some embodiments, the SpCas9 variants include one of the following clusters of mutations:
Mutation Cluster 1 N692A, M694A, Q695A, H698A
Mutation Cluster 2 G582A, V583A, E584A, D585A, N588A
Mutation Cluster 3 T657A, G658A, W659A, R661A
Mutation Cluster 4 F491A, M495A, T496A, N497A
Mutation Cluster 5 K918A, V922A, R925A, Q926A

Exemplary variants include those shown in Table A.

TABLE A

| | | Mutation # | | | | | | |
|---|---|---|---|---|---|---|---|---|
| Variant | name | 1 | 2 | 3 | 4 | 5 | 6 | Mutations |
| Cluster 1 | | N692A | M694A | Q695A | H698A | Q926A | — | N692A/M694A/Q695A/H698A/Q926A |
| Cluster 1 | A926Q | N692A | M694A | Q695A | H698A | — | — | N692A/M694A/Q695A/H698A |
| Cluster 1 | A698H | N692A | M694A | Q695A | — | Q926A | — | N692A/M694A/Q695A/Q926A |
| Cluster 1 | A695Q | N692A | M694A | — | H698A | Q926A | — | N692A/M694A/H698A/Q926A |
| Cluster 1 | A694M | N692A | — | Q695A | H698A | Q926A | — | N692A/Q695A/H698A/Q926A |
| Cluster 1 | A692N | — | M694A | Q695A | H698A | Q926A | — | M694A/Q695A/H698A/Q926A |
| Cluster 2 | | G582A | V583A | E584A | D585A | N588A | Q926A | G582A/V583A/E584A/D585A/N588A/Q926A |
| Cluster 2 | A926Q | G582A | V583A | E584A | D585A | N588A | — | G582A/V583A/E584A/D585A/N588A |
| Cluster 3 | | T657A | G658A | W659A | R661A | Q926A | — | T657A/G658A/W659A/R661A/Q926A |
| Cluster 3 | A926Q | T657A | G658A | W659A | R661A | — | — | T657A/G658A/W659A/R661A |
| Cluster 4 | | F491A | M495A | T496A | N497A | Q926A | — | F491A/M495A/T496A/N497A/Q926A |
| Cluster 4 | A926Q | F491A | M495A | T496A | N497A | — | — | F491A/M495A/T496A/N497A |
| Cluster 5 | | K918A | V922A | R925A | Q926A | — | — | K918A/V922A/R925A/Q926A |
| Cluster 5 | A926Q | K918A | V922A | R925A | — | — | — | K918A/V922A/R925A |

In some embodiments, the SpCas9 variants include one of the following sets of mutations: N692A/M694A/Q695A/H698A (we refer to the SpCas9 variant bearing all four of these mutations as Cluster 1 A926Q or HypaCas9).

In some embodiments, the SpCas9 variants also include one of the following mutations, which reduce or destroy the nuclease activity of the Cas9: D10, E762, D839, H983, or D986 and H840 or N863, e.g., D10A/D10N and H840A/H840N/H840Y, to render the nuclease portion of the protein catalytically inactive; substitutions at these positions could be alanine (as they are in Nishimasu al., Cell 156, 935-949 (2014)), or other residues, e.g., glutamine, asparagine, tyrosine, serine, or aspartate, e.g., E762Q, H983N, H983Y, D986N, N863D, N863S, or N863H (see WO 2014/152432). In some embodiments, the variant includes mutations at D10A or H840A (which creates a single-strand nickase), or mutations at D10A and H840A (which abrogates nuclease activity; this mutant is known as dead Cas9 or dCas9).

Also provided herein are isolated nucleic acids encoding the Cas9 variants, vectors comprising the isolated nucleic acids, optionally operably linked to one or more regulatory domains for expressing the variant proteins, and host cells, e.g., mammalian host cells, comprising the nucleic acids, and optionally expressing the variant proteins.

The variants described herein can be used for altering the genome of a cell; the methods generally include expressing the variant proteins in the cells, along with a guide RNA having a region complementary to a selected portion of the genome of the cell. Methods for selectively altering the genome of a cell are known in the art, see, e.g., U.S. Pat. No. 8,993,233; US 20140186958; U.S. Pat. No. 9,023,649; WO/2014/099744; WO 2014/089290; WO2014/144592; W0144288; WO2014/204578; WO2014/152432; WO2115/099850; U.S. Pat. No. 8,697,359; US20160024529; US20160024524; US20160024523; US20160024510; US20160017366; US20160017301; US20150376652; US20150356239; US20150315576; US20150291965; US20150252358; US20150247150; US20150232883; US20150232882; US20150203872; US20150191744; US20150184139; US20150176064; US20150167000; US20150166969; US20150159175; US20150159174; US20150093473; US20150079681; US20150067922; US20150056629; US20150044772; US20150024500; US20150024499; US20150020223; US20140356867; US20140295557; US20140273235; US20140273226; US20140273037; US20140189896; US20140113376; US20140093941; US20130330778; US20130288251; US20120088676; US20110300538; US20110236530; US20110217739; US20110002889; US20100076057; US20110189776; US20110223638; US20130130248; US20150050699; US20150071899; US20150050699; US20150045546; US20150031134; US20150024500; US20140377868; US20140357530; US20140349400; US20140335620; US20140335063; US20140315985; US20140310830; US20140310828; US20140309487; US20140304853; US20140298547; US20140295556; US20140294773; US20140287938; US20140273234; US20140273232; US20140273231; US20140273230; US20140271987; US20140256046; US20140248702; US20140242702; US20140242700; US20140242699; US20140242664; US20140234972; US20140227787; US20140212869; US20140201857; US20140199767; US20140189896; US20140186958; US20140186919; US20140186843; US20140179770; US20140179006; US20140170753; WO/2008/108989; WO/2010/054108; WO/2012/164565; WO/2013/098244; WO/2013/176772; US 20150071899; Makarova et al., “Evolution and classification of the CRISPR-Cas systems” 9(6) Nature Reviews Microbiology 467-477 (1-23) (June 2011); Wiedenheft et al., “RNA-guided genetic silencing systems in bacteria and archaea” 482 Nature 331-338 (Feb. 16, 2012); Gasiunas et al., “Cas9-crRNA ribonucleoprotein complex mediates specific DNA cleavage for adaptive immunity in bacteria” 109(39) Proceedings of the National Academy of Sciences USA E2579-E2586 (Sep. 4, 2012); Jinek et al., “A Programmable Dual-RNA-Guided DNA Endonuclease in Adaptive Bacterial Immunity” 337 Science 816-821 (Aug. 17, 2012); Carroll, “A CRISPR Approach to Gene Targeting” 20(9) Molecular Therapy 1658-1660 (September 2012); U.S. Appl. No. 61/652,086, filed May 25, 2012; Al-Attar et al., Clustered Regularly Interspaced Short Palindromic Repeats (CRISPRs): The Hallmark of an Ingenious Antiviral Defense Mechanism in Prokaryotes, Biol Chem. (2011) vol. 392, Issue 4, pp. 277-289; Hale et al., Essential Features and Rational Design of CRISPR RNAs That Function With the Cas RAMP Module Complex to Cleave RNAs, Molecular Cell, (2012) vol. 45, Issue 3, 292-302.

The variant proteins described herein can be used in place of or in addition to any of the Cas9 proteins described in the foregoing references, or in combination with mutations described therein. In addition, the variants described herein can be used in fusion proteins in place of the wild-type Cas9 or other Cas9 mutations (such as the dCas9 or Cas9 nickase described above) as known in the art, e.g., a fusion protein with a heterologous functional domains as described in U.S. Pat. No. 8,993,233; US 20140186958; U.S. Pat. No. 9,023,649; WO/2014/099744; WO 2014/089290; WO2014/144592; W0144288; WO2014/204578; WO2014/152432; WO2115/099850; U.S. Pat. No. 8,697,359; US2010/0076057; US2011/0189776; US2011/0223638; US2013/0130248; WO/2008/108989; WO/2010/054108; WO/2012/164565; WO/2013/098244; WO/2013/176772; US20150050699; US 20150071899 and WO 2014/124284. For example, the variants, preferably comprising one or more nuclease-reducing or killing mutation, can be fused on the N or C terminus of the Cas9 to a transcriptional activation domain or other heterologous functional domains (e.g., transcriptional repressors (e.g., KRAB, ERD, SID, and others, e.g., amino acids 473-530 of the ets2 repressor factor (ERF) repressor domain (ERD), amino acids 1-97 of the KRAB domain of KOX1, or amino acids 1-36 of the Mad mSIN3 interaction domain (SID); see Beerli et al., PNAS USA 95:14628-14633 (1998)) or silencers such as Heterochromatin Protein 1 (HP1, also known as swi6), e.g., HP1α or HP1β; proteins or peptides that could recruit long non-coding RNAs (lncRNAs) fused to a fixed RNA binding sequence such as those bound by the MS2 coat protein, endoribonuclease Csy4, or the lambda N protein; enzymes that modify the methylation state of DNA (e.g., DNA methyltransferase (DNMT) or TET proteins); or enzymes that modify histone subunits (e.g., histone acetyltransferases (HAT), histone deacetylases (HDAC), histone methyltransferases (e.g., for methylation of lysine or arginine residues) or histone demethylases (e.g., for demethylation of lysine or arginine residues)) as are known in the art can also be used. A number of sequences for such domains are known in the art, e.g., a domain that catalyzes hydroxylation of methylated cytosines in DNA. Exemplary proteins include the Ten-Eleven-Translocation (TET)1-3 family, enzymes that converts 5-methylcytosine (5-mC) to 5-hydroxymethylcytosine (5-hmC) in DNA.

Sequences for human TET1-3 are known in the art and are shown in the following table:

| | GenBank Accession Nos. | |
|---|---|---|
| Gene | Amino Acid | Nucleic Acid |
| TET 1 | NP_085128.2 | NM_030625.2 |
| TET2* | NP_001120680.1 (var 1) | NM_001127208.2 |
| | NP_060098.3 (var 2) | NM_017628.4 |
| TET3 | NP_659430.1 | NM_144993.1 |

*Variant (1) represents the longer transcript and encodes the longer isoform (a).
Variant (2) differs in the 5′UTR and in the 3′UTR and coding sequence compared to variant 1.
The resulting isoform (b) is shorter and has a distinct C-terminus compared to isoform a.

The present variants can also be used in “base editor” proteins, e.g., in place of the Cas9 protein in fusions of CRISPR/Cas9 and a deaminase, e.g., a cytidine deaminase enzyme, that retain the ability to be programmed with a guide RNA, do not induce dsDNA breaks, and mediate the direct conversion of cytidine to uridine, thereby effecting a C→T (or G→A) substitution, optionally with a uracil glycosylase inhibitor, as described in Komor et al., Nature. 2016 May 19; 533(7603):420-4, or a fusion protein containing a catalytically defective Cas9, a cytidine deaminase, and an inhibitor of base excision repair as described in Kim et al., Nat Biotechnol. 2017 April; 35(4):371-376. See, e.g., US 20170121693, In addition, the variants described herein can be used in dCas9-targeted somatic hypermutation methods that use catalytically inactive dCas9 used to recruit variants of cytidine deaminase (AID) with MS2-modified sgRNAs to specifically mutate endogenous targets with limited off-target damage as described in Hess et al., Nat Methods. 2016 December; 13(12):1036-1042.

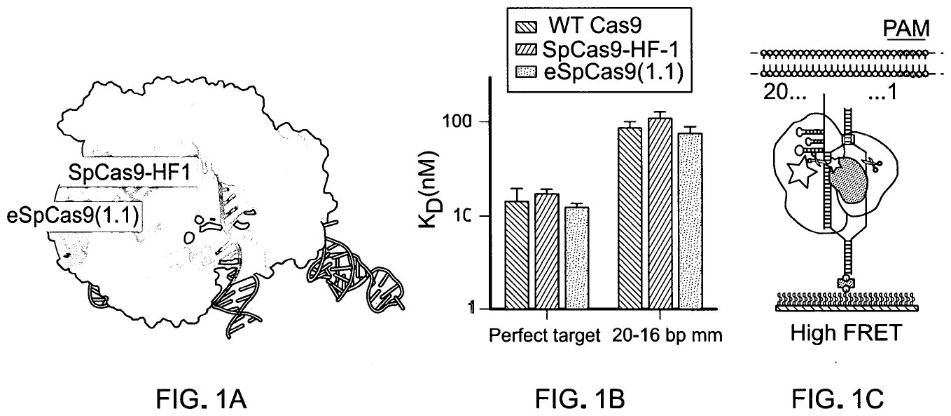

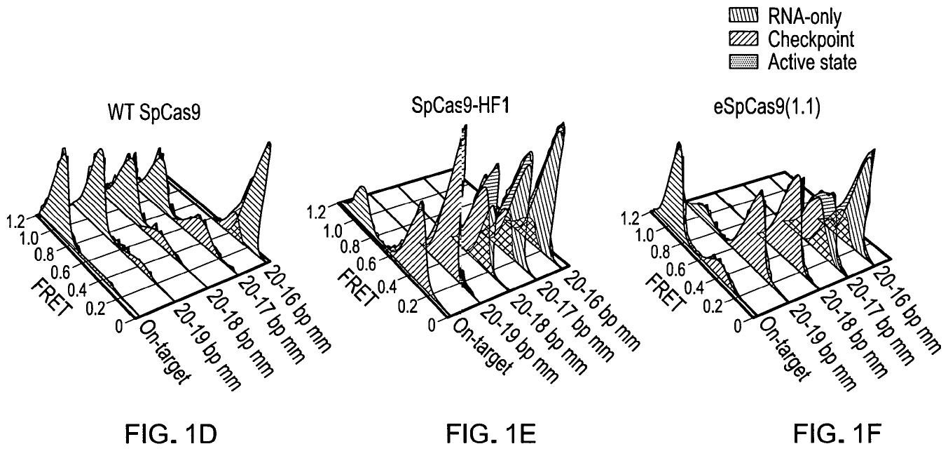

In some embodiments, all or part of the full-length sequence of the catalytic domain can be included, e.g., a catalytic module comprising the cysteine-rich extension and the 2OGFeDO domain encoded by 7 highly conserved exons, e.g., the Tet1 catalytic domain comprising amino acids 1580-2052, Tet2 comprising amino acids 1290-1905 and Tet3 comprising amino acids 966-1678. See, e.g., FIG. 1 of Iyer et al., Cell Cycle. 2009 Jun. 1; 8(11):1698-710. Epub 2009 Jun. 27, for an alignment illustrating the key catalytic residues in all three Tet proteins, and the supplementary materials thereof (available at ftp site ftp.ncbi.nih.gov/pub/aravind/DONS/supplementary_material_DONS.html) for full length sequences (see, e.g., seq 2c); in some embodiments, the sequence includes amino acids 1418-2136 of Tet1 or the corresponding region in Tet2/3.

Other catalytic modules can be from the proteins identified in Iyer et al., 2009.

In some embodiments, the heterologous functional domain is a biological tether, and comprises all or part of (e.g., DNA binding domain from) the MS2 coat protein, endoribonuclease Csy4, or the lambda N protein. These proteins can be used to recruit RNA molecules containing a specific stem-loop structure to a locale specified by the dCas9 gRNA targeting sequences. For example, a dCas9 variant fused to MS2 coat protein, endoribonuclease Csy4, or lambda N can be used to recruit a long non-coding RNA (lncRNA) such as XIST or HOTAIR; see, e.g., Keryer-Bibens et al., Biol. Cell 100:125-138 (2008), that is linked to the Csy4, MS2 or lambda N binding sequence. Alternatively, the Csy4, MS2 or lambda N protein binding sequence can be linked to another protein, e.g., as described in Keryer-Bibens et al., supra, and the protein can be targeted to the dCas9 variant binding site using the methods and compositions described herein. In some embodiments, the Csy4 is catalytically inactive. In some embodiments, the Cas9 variant, preferably a dCas9 variant, is fused to FokI as described in U.S. Pat. No. 8,993,233; US 20140186958; U.S. Pat. No. 9,023,649; WO/2014/099744; WO 2014/089290; WO2014/144592; W0144288; WO2014/204578; WO2014/152432; WO2115/099850; U.S. Pat. No. 8,697,359; US2010/0076057; US2011/0189776; US2011/0223638; US2013/0130248; WO/2008/108989; WO/2010/054108; WO/2012/164565; WO/2013/098244; WO/2013/176772; US20150050699; US 20150071899 and WO 2014/204578.

In some embodiments, the fusion proteins include a linker between the dCas9 variant and the heterologous functional domains. Linkers that can be used in these fusion proteins (or between fusion proteins in a concatenated structure) can include any sequence that does not interfere with the function of the fusion proteins. In preferred embodiments, the linkers are short, e.g., 2-20 amino acids, and are typically flexible (i.e., comprising amino acids with a high degree of freedom such as glycine, alanine, and serine). In some embodiments, the linker comprises one or more units consisting of GGGS (SEQ ID NO:2) or GGGGS (SEQ ID NO:3), e.g., two, three, four, or more repeats of the GGGS (SEQ ID NO:2) or GGGGS (SEQ ID NO:3) unit. Other linker sequences can also be used.

In some embodiments, the variant protein includes a cell-penetrating peptide sequence that facilitates delivery to the intracellular space, e.g., HIV-derived TAT peptide, penetratins, transportans, or hCT derived cell-penetrating peptides, see, e.g., Caron et al., (2001) Mol Ther. 3(3):310-8; Langel, Cell-Penetrating Peptides: Processes and Applications (CRC Press, Boca Raton Fla. 2002); El-Andaloussi et al., (2005) Curr Pharm Des. 11(28):3597-611; and Deshayes et al., (2005) Cell Mol Life Sci. 62(16):1839-49.

Cell penetrating peptides (CPPs) are short peptides that facilitate the movement of a wide range of biomolecules across the cell membrane into the cytoplasm or other organelles, e.g. the mitochondria and the nucleus. Examples of molecules that can be delivered by CPPs include therapeutic drugs, plasmid DNA, oligonucleotides, siRNA, peptide-nucleic acid (PNA), proteins, peptides, nanoparticles, and liposomes. CPPs are generally 30 amino acids or less, are derived from naturally or non-naturally occurring protein or chimeric sequences, and contain either a high relative abundance of positively charged amino acids, e.g. lysine or arginine, or an alternating pattern of polar and non-polar amino acids. CPPs that are commonly used in the art include Tat (Frankel et al., (1988) Cell. 55:1189-1193, Vives et al., (1997) J. Biol. Chem. 272:16010-16017), penetratin (Derossi et al., (1994) J. Biol. Chem. 269:10444-10450), polyarginine peptide sequences (Wender et al., (2000) Proc. Natl. Acad. Sci. USA 97:13003-13008, Futaki et al., (2001) J. Biol. Chem. 276:5836-5840), and transportan (Pooga et al., (1998) Nat. Biotechnol. 16:857-861).

CPPs can be linked with their cargo through covalent or non-covalent strategies. Methods for covalently joining a CPP and its cargo are known in the art, e.g. chemical cross-linking (Stetsenko et al., (2000) J. Org. Chem. 65:4900-4909, Gait et al. (2003) Cell. Mol. Life. Sci. 60:844-853) or cloning a fusion protein (Nagahara et al., (1998) Nat. Med. 4:1449-1453). Non-covalent coupling between the cargo and short amphipathic CPPs comprising polar and non-polar domains is established through electrostatic and hydrophobic interactions.

CPPs have been utilized in the art to deliver potentially therapeutic biomolecules into cells. Examples include cyclosporine linked to polyarginine for immunosuppression (Rothbard et al., (2000) Nature Medicine 6(11):1253-1257), siRNA against cyclin B1 linked to a CPP called MPG for inhibiting tumorigenesis (Crombez et al., (2007) Biochem Soc. Trans. 35:44-46), tumor suppressor p53 peptides linked to CPPs to reduce cancer cell growth (Takenobu et al., (2002) Mol. Cancer Ther. 1(12):1043-1049, Snyder et al., (2004) PLoS Biol. 2:E36), and dominant negative forms of Ras or phosphoinositol 3 kinase (PI3K) fused to Tat to treat asthma (Myou et al., (2003) J. Immunol. 171:4399-4405).

CPPs have been utilized in the art to transport contrast agents into cells for imaging and biosensing applications. For example, green fluorescent protein (GFP) attached to Tat has been used to label cancer cells (Shokolenko et al., (2005) DNA Repair 4(4):511-518). Tat conjugated to quantum dots have been used to successfully cross the blood-brain barrier for visualization of the rat brain (Santra et al., (2005) Chem. Commun. 3144-3146). CPPs have also been combined with magnetic resonance imaging techniques for cell imaging (Liu et al., (2006) Biochem. and Biophys. Res. Comm. 347(1):133-140). See also Ramsey and Flynn, Pharmacol Ther. 2015 Jul. 22. pii: S0163-7258(15)00141-2.

Alternatively or in addition, the variant proteins can include a nuclear localization sequence, e.g., SV40 large T antigen NLS (PKKKRRV (SEQ ID NO:4)) and nucleoplasmin NLS (KRPAATKKAGQAKKKK (SEQ ID NO:5)). Other NLSs are known in the art; see, e.g., Cokol et al., EMBO Rep. 2000 Nov. 15; 1(5): 411-415; Freitas and Cunha, Curr Genomics. 2009 December; 10(8): 550-557.

In some embodiments, the variants include a moiety that has a high affinity for a ligand, for example GST, FLAG or hexahistidine sequences. Such affinity tags can facilitate the purification of recombinant variant proteins.

For methods in which the variant proteins are delivered to cells, the proteins can be produced using any method known in the art, e.g., by in vitro translation, or expression in a suitable host cell from nucleic acid encoding the variant protein; a number of methods are known in the art for producing proteins. For example, the proteins can be produced in and purified from yeast, E. coli, insect cell lines, plants, transgenic animals, or cultured mammalian cells; see, e.g., Palomares et al., “Production of Recombinant Proteins: Challenges and Solutions,” Methods Mol Biol. 2004; 267:15-52. In addition, the variant proteins can be linked to a moiety that facilitates transfer into a cell, e.g., a lipid nanoparticle, optionally with a linker that is cleaved once the protein is inside the cell. See, e.g., LaFountaine et al., Int J Pharm. 2015 Aug. 13; 494(1):180-194.

Expression Systems

To use the Cas9 variants described herein, it may be desirable to express them from a nucleic acid that encodes them. This can be performed in a variety of ways. For example, the nucleic acid encoding the Cas9 variant can be cloned into an intermediate vector for transformation into prokaryotic or eukaryotic cells for replication and/or expression. Intermediate vectors are typically prokaryote vectors, e.g., plasmids, or shuttle vectors, or insect vectors, for storage or manipulation of the nucleic acid encoding the Cas9 variant for production of the Cas9 variant. The nucleic acid encoding the Cas9 variant can also be cloned into an expression vector, for administration to a plant cell, animal cell, preferably a mammalian cell or a human cell, fungal cell, bacterial cell, or protozoan cell.

To obtain expression, a sequence encoding a Cas9 variant is typically subcloned into an expression vector that contains a promoter to direct transcription. Suitable bacterial and eukaryotic promoters are well known in the art and described, e.g., in Sambrook et al., Molecular Cloning, A Laboratory Manual (3d ed. 2001); Kriegler, Gene Transfer and Expression: A Laboratory Manual (1990); and Current Protocols in Molecular Biology (Ausubel et al., eds., 2010). Bacterial expression systems for expressing the engineered protein are available in, e.g., E. coli, Bacillus sp., and Salmonella (Palva et al., 1983, Gene 22:229-235). Kits for such expression systems are commercially available. Eukaryotic expression systems for mammalian cells, yeast, and insect cells are well known in the art and are also commercially available.

The promoter used to direct expression of a nucleic acid depends on the particular application. For example, a strong constitutive promoter is typically used for expression and purification of fusion proteins. In contrast, when the Cas9 variant is to be administered in vivo for gene regulation, either a constitutive or an inducible promoter can be used, depending on the particular use of the Cas9 variant. In addition, a preferred promoter for administration of the Cas9 variant can be a weak promoter, such as HSV TK or a promoter having similar activity. The promoter can also include elements that are responsive to transactivation, e.g., hypoxia response elements, Gal4 response elements, lac repressor response element, and small molecule control systems such as tetracycline-regulated systems and the RU-486 system (see, e.g., Gossen & Bujard, 1992, Proc. Natl. Acad. Sci. USA, 89:5547; Oligino et al., 1998, Gene Ther., 5:491-496; Wang et al., 1997, Gene Ther., 4:432-441; Neering et al., 1996, Blood, 88:1147-55; and Rendahl et al., 1998, Nat. Biotechnol., 16:757-761).

In addition to the promoter, the expression vector typically contains a transcription unit or expression cassette that contains all the additional elements required for the expression of the nucleic acid in host cells, either prokaryotic or eukaryotic. A typical expression cassette thus contains a promoter operably linked, e.g., to the nucleic acid sequence encoding the Cas9 variant, and any signals required, e.g., for efficient polyadenylation of the transcript, transcriptional termination, ribosome binding sites, or translation termination. Additional elements of the cassette may include, e.g., enhancers, and heterologous spliced intronic signals.

The particular expression vector used to transport the genetic information into the cell is selected with regard to the intended use of the Cas9 variant, e.g., expression in plants, animals, bacteria, fungus, protozoa, etc. Standard bacterial expression vectors include plasmids such as pBR322 based plasmids, pSKF, pET23D, and commercially available tag-fusion expression systems such as GST and LacZ.

Expression vectors containing regulatory elements from eukaryotic viruses are often used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells.

The vectors for expressing the Cas9 variants can include RNA Pol III promoters to drive expression of the guide RNAs, e.g., the H1, U6 or 7SK promoters. These human promoters allow for expression of Cas9 variants in mammalian cells following plasmid transfection.

Some expression systems have markers for selection of stably transfected cell lines such as thymidine kinase, hygromycin B phosphotransferase, and dihydrofolate reductase. High yield expression systems are also suitable, such as using a baculovirus vector in insect cells, with the gRNA encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of recombinant sequences.

Standard transfection methods are used to produce bacterial, mammalian, yeast or insect cell lines that express large quantities of protein, which are then purified using standard techniques (see, e.g., Colley et al., 1989, J. Biol. Chem., 264:17619-22; Guide to Protein Purification, in Methods in Enzymology, vol. 182 (Deutscher, ed., 1990)). Transformation of eukaryotic and prokaryotic cells are performed according to standard techniques (see, e.g., Morrison, 1977, J. Bacteriol. 132:349-351; Clark-Curtiss & Curtiss, Methods in Enzymology 101:347-362 (Wu et al., eds, 1983).

Any of the known procedures for introducing foreign nucleotide sequences into host cells may be used. These include the use of calcium phosphate transfection, polybrene, protoplast fusion, electroporation, nucleofection, liposomes, microinjection, naked DNA, plasmid vectors, viral vectors, both episomal and integrative, and any of the other well-known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the Cas9 variant. In some embodiments, the variants are delivered via RNP delivery (delivering purified protein pre-complexed with the guide RNA).

The present invention also includes the vectors and cells comprising the vectors, as well as kits comprising the proteins and nucleic acids described herein, e.g., for use in a method described herein.

### EXAMPLES

The invention is further described in the following examples, which do not limit the scope of the invention described in the claims.

### Example 1. Conformational Gating Drives CRISPR-Cas9 Targeting Accuracy

Methods

Protein Purification and Dye Labeling.

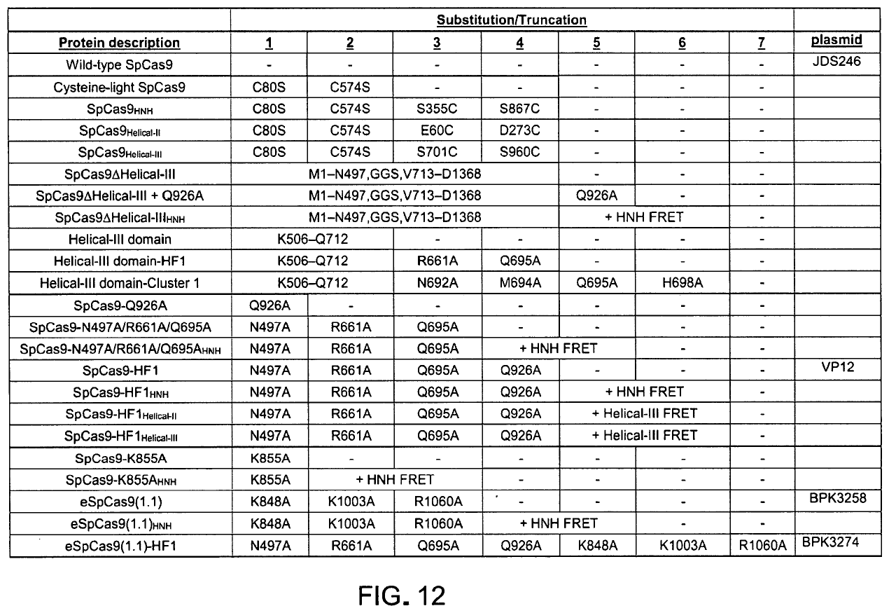

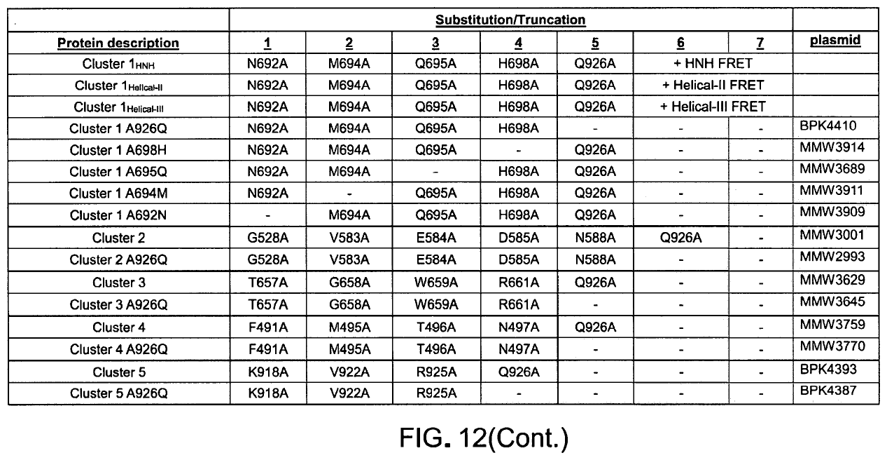

S. pyogenes Cas9 and truncation derivatives were cloned into a custom pET-based expression vector containing an N-terminal Hisio-tag, maltose-binding protein (MBP) and TEV protease cleavage site. Point mutations were introduced by Gibson assembly or around-the-horn PCR and verified by DNA sequencing. Proteins were purified as described¹⁸, with the following modifications: after Ni-NTA affinity purification and overnight TEV cleavage at 4° C., proteins were purified over an MBPTrap HP column connected to a HiTrap Heparin HP column for cation exchange chromatography. The final gel filtration step (Superdex 200) was carried out in elution buffer containing 20 mM Tris-HCl pH 7.5, 200 mM NaCl, 5% glycerol and 1 mM TCEP. For FRET experiments, dye-labeled Cas9 samples were prepared as described¹⁰. A list of all protein variants and truncations are listed in FIG. 12.

Human Expression Plasmids for FIG. 12

| | Addgene | |
|---|---|---|
| Name | # | Description |
| JDS246 | 43861 | pCMV-T7-hSpCas9-NLS(SV40)-3xFLAG |
| VP12 | 72247 | pCMV-T7-hSpCas9-HF1(N497A/R661A/Q695A/Q926A)- |
| | | NLS(SV40)-3xFLAG |
| BPK3258 | | pCMV-T7-heSpCas9(1.1)(K848A/K1003A/R1060A)- |
| | | NLS(SV40)-3xFLAG |
| BPK3274 | | pCMV-T7-heSpCas9(1.1)- |
| | | HF1(N497A/R661A/Q695A/K848A/Q926A/K1003A/R1060A)- |
| | | NLS(SV40)-3xFLAG |
| MMW3709 | | pCMV-T7-hSpCas9- |
| | | Cluster1(N692A/M694A/Q695A/H698A/Q926A)-NLS(SV40)- |
| | | 3xFLAG |
| BPK4410 | | pCMV-T7-hSpCas9-Cluster1- |
| | | A926Q(N692A/M694A/Q695A/H698A)-NLS(SV40)-3xFLAG |
| MMW3914 | | pCMV-T7-hSpCas9-Cluster1- |
| | | 698H(N692A/M694A/Q695A/Q926A)-NLS(SV40)-3xFLAG |
| MMW3689 | | pCMV-T7-hSpCas9-Cluster1- |
| | | A695Q(N692A/M694A/H698A/Q926A)-NLS(SV40)-3xFLAG |
| MMW3911 | | pCMV-T7-hSpCas9-Cluster1- |
| | | A694M(N692A/Q695A/H698A/Q926A)-NLS(SV40)-3xFLAG |
| MMW3909 | | pCMV-T7-hSpCas9-Cluster1- |
| | | A692N(M694A/Q695A/H698A/Q926A)-NLS(SV40)-3xFLAG |
| MMW3001 | | pCMV-T7-hSpCas9- |
| | | Cluster2(G528A/V583A/E584A/D585A/N588A/Q926A)- |
| | | NLS(SV40)-3xFLAG |
| MMW2993 | | pCMV-T7-hSpCas9-Cluster2- |
| | | A926Q(G528A/V583A/E584A/D585A/N588A)-NLS(SV40)- |
| | | 3xFLAG |
| MMW3629 | | pCMV-T7-hSpCas9- |
| | | Cluster3(T657A/G658A/W659A/R661A/Q926A)-NLS(SV40)- |
| | | 3xFLAG |
| MMW3645 | | pCMV-T7-hSpCas9-Cluster3- |
| | | A926Q(T657A/G658A/W659A/R661A)-NLS(SV40)-3xFLAG |
| MMW3759 | | pCMV-T7-hSpCas9- |
| | | Cluster4(F491A/M495A/T496A/N497A/Q926A)-NLS(SV40)- |
| | | 3xFLAG |
| MMW3770 | | pCMV-T7-hSpCas9-Cluster4- |
| | | A926Q(F491A/M495A/T496A/N497A)-NLS(SV40)-3xFLAG |
| BPK4393 | | pCMV-T7-hSpCas9-Cluster5(K918A/V922A/R925A/Q926A)- |
| | | NLS(SV40)-3xFLAG |
| BPK4387 | | pCMV-T7-hSpCas9-Cluster5-A926Q(K918A/V922A/R925A)- |
| | | NLS(SV40)-3xFLAG |

Nucleic Acid Preparation.

sgRNA templates were PCR amplified from a pUC19 vector containing a T7 promoter, 20 nt target sequence and optimized sgRNA scaffold. The amplified PCR product was extracted with phenol:chloroform:isoamylalcohol and served as the DNA template for sgRNA transcription reactions, which were performed as described¹⁹. DNA oligonucleotides and 5′end biotinylated DNAs (Table 1) were ordered synthetically (Integrated DNA Technologies), and DNA duplexes were prepared and purified by native PAGE as described¹⁸.

DNA Cleavage and Binding Assays.

DNA duplex substrates were 5′-[³²P]-radiolabeled on both strands. For cleavage experiments, Cas9 and sgRNA were pre-incubated at room temperature for at least 10 min in 1× binding buffer (20 mM Tris-HCl pH 7.5, 100 mM KCl, 5 mM MgCl₂, 1 mM DTT, 5% glycerol, 50 μg/ml heparin) before initiating the cleavage reaction by addition of DNA duplexes. For Helical-III domain add-back experiments, Cas9 and sgRNA were pre-incubated with 10-fold molar excess of Helical-III for at least 10 minutes at room temperature before addition of radiolabeled substrate. DNA cleavage experiments were performed and analyzed as previously described¹⁰. DNA binding assays were conducted in 1× binding buffer without MgCl₂+1 mM EDTA at room temperature for 2 hours. DNA-bound complexes were resolved on 8% native PAGE (0.5×TBE+1 mM EDTA, without MgCl₂) at 4° C., as previously described⁹. Experiments were replicated at least three times, and presented gels are representative results.

Bulk FRET Experiments.

All bulk FRET assays were performed at room temperature in 1× binding buffer, containing 50 nM SpCas9_{HNH} (C80S/S355C/C574S/S867C labeled with Cy3/Cy5) or SpCas9_{Helical-II} (E60C/C80S/D273C/C574S labeled with Cy3/Cy5) with 200 nM sgRNA and DNA substrate where indicated. Fluorescence measurements were collected and analyzed as described¹⁰.

Sample Preparation for smFRET Assay.

Quartz slides coated with 99% PEG and 1% biotinylated-PEG was acquired from MicroSurfaces, Inc. An air-tight sample chamber was prepared by sandwiching double-sided tape between quartz slides and coverslips. To prepare the slides for molecule deposition, the PEG surface was pre-blocked with 10 mg/ml casein incubated for 10 min. The flow chamber was then washed with 1× binding buffer and then incubated with 20 μl 1 mg/ml streptavidin for 10 min. Excess streptavidin was washed away with 40 μl 1× binding buffer. To surface immobilize Cas9 from its DNA substrate, 1 nM biotinylated-DNA substrate (NTS (5′biotin) hybridized to TS) was flown into the sample chamber and incubated for 10 min. The chamber was washed with 1× binding buffer. 50 nM Cas9 and 50 nM sgRNA were mixed in 1× binding buffer and incubated for 10 min. Sample was spun at 16,000×g at 4° C. for 5 minutes. The supernatant was diluted to 100 pM, flown into sample chamber and incubated for 10 min. Before data acquisition, the sample chamber was washed with 1× binding buffer and 20 μL imaging buffer (1 mg/ml glucose oxidase, 0.04 mg/ml catalase, 0.8% dextrose and 2 mM Trolox in 1× binding buffer).

Microscopy and Data Analysis.

A prism-type TIRF microscope was setup using a Nikon Ti-E Eclipse inverted fluorescent microscope equipped with a 60×1.20 N.A. Plan Apo water objective and the perfect focusing system (Nikon). A 532-nm solid state laser (Coherent Compass) and a 633-nm HeNe laser (JDSU) were used for Cy3 and Cy5 excitation, respectively. Cy3 and Cy5 fluorescence were split into two channels using an Optosplit II image splitter (Cairn Instruments) and imaged separately on the same electron-multiplied charged-coupled device (EM-CCD) camera (512×512 pixels, Andor Ixon EM⁺). Effective pixel size of the camera was set to 267 nm after magnification. Movies for steady-state FRET measurements were acquired at 10 Hz under 0.3 kW cm⁻² 532-nm excitation.

Human Cell Culture and Transfection.

Descriptions of nuclease and guide RNA plasmids used for human cell culture are available in Tables 1 and 2, respectively. Nuclease variants were generated by isothermal assembly into JDS246 (Addgene #43861)⁴, and guide RNAs were cloned into BsmBI digested BPK1520 (Addgene #65777)²⁰. Both U2OS cells (a gift from Toni Cathomen, Freiburg) and U2OS-EGFP cells (encoding a single integrated copy of a pCMV-EGFP-PEST cassette)²¹ were cultured at 37° C. with 5% CO₂ in advanced DMEM containing 10% heat-inactivated fetal bovine serum, 2 mM GlutaMax, penicillin/streptomycin, and 400 μg ml⁻¹ Geneticin (for U20S-EGFP cells only). Cell culture reagents were purchased from Thermo Fisher Scientific, cell line identities were validated by STR profiling (ATCC) and deep-sequencing, and cell culture supernatant was tested bi-weekly for mycoplasma. Transfections were performed using a Lonza 4-D Nucleofector with the SE Kit and the DN-100 program on ˜200 k cells with 750 ng of nuclease and 250 ng of nuclease and guide RNA plasmids, respectively.

Human Cell EGFP Disruption Assay.

EGFP disruption experiments were performed as previously described^{4,21}. Briefly, transfected cells were analyzed ˜52 hours post-transfection for loss of EGFP fluorescence using a Fortessa flow cytometer (BD Biosciences). Background loss was determined by gating a negative control transfection (containing nuclease and empty guide RNA plasmid) at ˜2.5% for all experiments.

T7 Endonuclease I Assays.

Roughly 72 hours post-transfection, genomic DNA was extracted from U2OS cells using the Agencourt DNAdvance Genomic DNA Isolation Kit (Beckman Coulter Genomics), and T7 endonuclease I assays were performed as previously described²¹. Briefly, 600-800 nt amplicons surrounding on-target sites were amplified from ˜100 ng of genomic DNA using Phusion Hot-Start Flex DNA Polymerase (New England Biolabs) using the primers listed in Supplementary Table 2. PCR products were visualized (using a QIAxcel capillary electrophoresis instrument, Qiagen), and purified (Agencourt Ampure XP cleanup, Beckman Coulter Genomics), Denaturation and annealing of ˜200 ng of the PCR product was followed by digestion with T7 endonuclease I (New England Biolabs). Digestion products were purified (Ampure) and quantified (QIAxcel) to approximate the mutagenesis frequencies induced by Cas9-sgRNA complexes.

TABLE 1
Sequences of all nucleic acids used in the study

| | | SEQ ID |
|---|---|---|
| | | NO: |
| in vitro DNA | | |
| substrates | sequence | |
| lambda1-targeting | GACGCATAAAGATGAGACGCGTTTTAGAGCTATGCTGTTTTG | 6. |
| sgRNA | GAAACAAAACAGCATAGCAAGTTAAAATAAGGCTAGTCCGTT | |
| | ATCAACTTGAAAAAGTGGCACCGAGTCGGTGC | |
| lambda1 TS on-target | AGCTGACGTTTGTACTCCAGCGTCTCATCTTTATGCGTCAGC | 7. |
| | AGAGATTTCTGCT | |
| lambda1 NTS on-target | AGCAGAAATCTCTGCTGACGCATAAAGATGAGACGCTGGAGT | 8. |
| | ACAAACGTCAGCT | |
| lambda1 NTS | /5Biosg/AGCAGAAATCTCTGCTGACGCATAAAGATGAGAC | 9. |
| (5′biotin) | GCTGGAGTACAAACGTCAGCT | |
| lambda1 TS 1-bp mm | AGCTGACGTTTGTACTCCAGCGTCTCATCTTTATGCGTGAGC | 10. |
| | AGAGATTTCTGCT | |
| lambda1 NTS 1-bp mm | AGCAGAAATCTCTGCTCACGCATAAAGATGAGACGCTGGAGT | 11. |
| | ACAAACGTCAGCT | |
| lambda1 NTS | /5Biosg/AGCAGAAATCTCTGCTCACGCATAAAGATGAGAC | 12. |
| (5′biotin) 1-bp mm | GCTGGAGTACAAACGTCAGCT | |
| lambda1 TS 2-bp mm | AGCTGACGTTTGTACTCCAGCGTCTCATCTTTATGCGAGAGC | 13. |
| | AGAGATTTCTGCT | |
| lambda1 NTS 2-bp mm | AGCAGAAATCTCTGCTCTCGCATAAAGATGAGACGCTGGAGT | 14. |
| | ACAAACGTCAGCT | |
| lambda1 NTS | /5Biosg/AGCAGAAATCTCTGCTCTCGCATAAAGATGAGAC | 15. |
| (5′biotin) 2-bp mm | GCTGGAGTACAAACGTCAGCT | |
| lambda1 TS 3-bp mm | AGCTGACGTTTGTACTCCAGCGTCTCATCTTTATGCCAGAGC | 16. |
| | AGAGATTTCTGCT | |
| lambda1 NTS 3-bp mm | AGCAGAAATCTCTGCTCTGGCATAAAGATGAGACGCTGGAGT | 17. |
| | ACAAACGTCAGCT | |
| lambda1 NTS | /5Biosg/AGCAGAAATCTCTGCTCTGGCATAAAGATGAGAC | 18. |
| (5′biotin) 3-bp mm | GCTGGAGTACAAACGTCAGCT | |
| lambda1 TS 4-bp mm | AGCTGACGTTTGTACTCCAGCGTCTCATCTTTATGGCAGAGC | 19. |
| | AGAGATTTCTGCT | |
| lambda1 NTS 4-bp mm | AGCAGAAATCTCTGCTCTGCCATAAAGATGAGACGCTGGAGT | 20. |
| | ACAAACGTCAGCT | |
| lambda1 NTS | /5Biosg/AGCAGAAATCTCTGCTCTGCCATAAAGATGAGAC | 21. |
| (5′biotin) 4-bp mm | GCTGGAGTACAAACGTCAGCT | |
| T7E1 primer sequence | T7E1 primer description | |
| CCAGAATGCACAAAGTACTGC | forward primer to amplify DNMT1 target | 22. |
| AC | sites in U2OS human cells | |
| GCCAAAGCCCGAGAGAGTGCC | reverse primer to amplify DNMT1 target | 23. |
| | sites in U2OS human cells | |
| GGAGCAGCTGGTCAGAGGGG | forward primer to amplify EMX1 target | 24. |
| | sites in U2OS human cells | |
| CCATAGGGAAGGGGGACACTG | reverse primer to amplify EMX1 target | 25. |
| G | sites in U2OS human cells | |
| GGGCCGGGAAAGAGTTGCTG | forward primer to amplify FANCF target | 26. |
| | sites in U2OS human cells (set #1) | |
| GCCCTACATCTGCTCTCCCTC | reverse primer to amplify FANCF target | 27. |
| C | sites in U2OS human cells (set #1) | |
| CAGCATGTGCACCGCAGACC | forward primer to amplify FANCF target | 28. |
| | sites in U2OS human cells (set #2) | |
| TCATCTCGCACGTGGTTCCGG | reverse primer to amplify FANCF target | 29. |
| | sites in U2OS human cells (set #2) | |
| CCAGCACAACTTACTCGCACT | forward primer to amplify RUNX1 target | 30. |
| TGAC | sites in U2OS human cells | |
| CATCACCAACCCACAGCCAAG | reverse primer to amplify RUNX1 target | 31. |
| G | sites in U2OS human cells | |
| ATCCCTGGACACTTCCCAAAG | forward primer to amplify VEGFA target | 32. |
| GAC | sites 1 and 3 in U2OS human cells | |
| CTCGACCCCCACCAAGGTTCA | reverse primer to amplify VEGFA target | 33. |
| C | sites 1 and 3 in U2OS human cells | |
| CGAGGAAGAGAGAGACGGGGT | forward primer to amplify VEGFA target | 34. |
| C | site 2 in U2OS human cells | |
| CTCCAATGCACCCAAGACAGC | reverse primer to amplify VEGFA target | 35. |
| AG | site 2 in U2OS human cells | |
| AGTGTGGGGTGTGTGGGAAG | forward primer to amplify ZSCAN2 target | 36. |
| | sites in U2OS human cells | |
| ACGGGACTTGACTCAGACCAC | reverse primer to amplify ZSCAN2 target | 37. |
| T | sites in U2OS human cells | |

sgRNAs against EGFP

| EGFP sgRNA plasmid | | |
|---|---|---|
| description | target sequence with PAM | |
| EGFP NGG site 1 | GGGCACGGGCAGCTTGCCGGTGG | 38. |
| EGFP NGG site 2 | GTCGCCCTCGAACTTCACCTCGG | 39. |
| EGFP NGG site 3 | GTAGGTCAGGGTGGTCACGAGGG | 40. |
| EGFP NGG site 4 | GGCGAGGGCGATGCCACCTACGG | 41. |
| EGFP NGG site 5 | GGTCGCCACCATGGTGAGCAAGG | 42. |
| EGFP NGG site 6 | GGTCAGGGTGGTCACGAGGGTGG | 43. |
| EGFP NGG site 7 | GGTGGTGCAGATGAACTTCAGGG | 44. |
| EGFP NGG site 8 | GTTGGGGTCTTTGCTCAGGGCGG | 45. |
| EGFP NGG site 9 | GGTGGTCACGAGGGTGGGCCAGG | 46. |
| EGFP NGG site 10 | GATGCCGTTCTTCTGCTTGTCGG | 47. |
| EGFP NGG site 11 | GTCGCCACCATGGTGAGCAAGGG | 48. |
| EGFP NGG site 12 | GCACTGCACGCCGTAGGTCAGGG | 49. |
| EGFP NGG site 1, | GGGCACGGGCAGCTTGCCccTGG | 50. |
| mismatches at 1 & 2 | | |
| EGFP NGG site 1, | GGGCACGGGCAGCTTGggGGTGG | 51. |
| mismatches at 3 & 4 | | |
| EGFP NGG site 1, | GGGCACGGGCAGCTacCCGGTGG | 52. |
| mismatches at 5 & 6 | | |
| EGFP NGG site 1, | GGGCACGGGCAGgaTGCCGGTGG | 53. |
| mismatches at 7 & 8 | | |
| EGFP NGG site 1, | GGGCACGGGCtcCTTGCCGGTGG | 54. |
| mismatches at 9 & 10 | | |
| EGFP NGG site 1, | GGGCACGGcgAGCTTGCCGGTGG | 55. |
| mismatches at 11 & 12 | | |
| EGFP NGG site 1, | GGGCACccGCAGCTTGCCGGTGG | 56. |
| mismatches at 13 & 14 | | |
| EGFP NGG site 1, | GGGCtgGGGCAGCTTGCCGGTGG | 57. |
| mismatches at 15 & 16 | | |
| EGFP NGG site 1, | GGcgACGGGCAGCTTGCCGGTGG | 58. |
| mismatches at 17 & 18 | | |
| EGFP NGG site 1, | GccCACGGGCAGCTTGCCGGTGG | 59. |
| mismatches at 18 & 19 | | |
| EGFP NGG site 7, | GGTGGTGCAGATGAACTTgtGGG | 60. |
| mismatches at 1 & 2 | | |
| EGFP NGG site 7, | GGTGGTGCAGATGAACaaCAGGG | 61. |
| mismatches at 3 & 4 | | |
| EGFP NGG site 7, | GGTGGTGCAGATGAtgTTCAGGG | 62. |
| mismatches at 5 & 6 | | |
| EGFP NGG site 7, | GGTGGTGCAGATctACTTCAGGG | 63. |
| mismatches at 7 & 8 | | |
| EGFP NGG site 7, | GGTGGTGCAGtaGAACTTCAGGG | 64. |
| mismatches at 9 & 10 | | |
| EGFP NGG site 7, | GGTGGTGCtcATGAACTTCAGGG | 65. |
| mismatches at 11 & 12 | | |
| EGFP NGG site 7, | GGTGGTcgAGATGAACTTCAGGG | 66. |
| mismatches at 13 & 14 | | |
| EGFP NGG site 7, | GGTGcaGCAGATGAACTTCAGGG | 67. |
| mismatches at 15 & 16 | | |
| EGFP NGG site 7, | GGacGTGCAGATGAACTTCAGGG | 68. |
| mismatches at 17 & 18 | | |
| EGFP NGG site 7, | GcaGGTGCAGATGAACTTCAGGG | 69. |
| mismatches at 18 & 19 | | |

sgRNAs against endogenous sites

| endogenous sgRNA | | |
|---|---|---|
| plasmid description | target sequence with PAM | |
| DNMT1 NGG site 1 | GTCACTCTGGGGAACACGCCCGG | 70. |
| DNMT1 NGG site 2 | GAGTGCTAAGGGAACGTTCACGG | 71. |
| DNMT1 NGG site 3 | GAGACTGAACACTCCTCAAACGG | 72. |
| DNMT1 NGG site 4 | GGAGTGAGGGAAACGGCCCCAGG | 73. |
| EMX1 NGG site 1 | GAGTCCGAGCAGAAGAAGAAGGG | 74. |
| EMX1 NGG site 2 | GTCACCTCCAATGACTAGGGTGG | 75. |
| EMX1 NGG site 3 | GGGAAGACTGAGGCTACATAGGG | 76. |
| FANCF NGG site 1 | GGAATCCCTTCTGCAGCACCTGG | 77. |
| FANCF NGG site 2 | GCTGCAGAAGGGATTCCATGAGG | 78. |
| FANCF NGG site 3 | GGCGGCTGCACAACCAGTGGAGG | 79. |
| FANCF NGG site 4 | GCTCCAGAGCCGTGCGAATGGGG | 80. |
| FANCF NGG site 5 | GAAGCTCGGAAAAGCGATCCAGG | 81. |
| FANCF NGG site 6 | GCTTGAGACCGCCAGAAGCTCGG | 82. |
| FANCF NGG site 7 | GACCAAAGCGCCGATGGATGTGG | 83. |
| FANCF NGG site 8 | GGGGTCCCAGGTGCTGACGTAGG | 84. |
| FANCF NGG site 9 | GGATCGCTTTTCCGAGCTTCTGG | 85. |
| FANCF NGG site 10 | GGATTCCATGAGGTGCGCGAAGG | 86. |
| FANCF NGG site 11 | GCGACTCTCTGCGTACTGATTGG | 87. |
| RUNX1 NGG site 1 | GCATTTTCAGGAGGAAGCGATGG | 88. |
| RUNX1 NGG site 2 | GGGAGAAGAAAGAGAGATGTAGG | 89. |
| VEGFA NGG site 1 | GGGTGGGGGGAGTTTGCTCCTGG | 90. |
| VEGFA NGG site 2 | GACCCCCTCCACCCCGCCTCCGG | 91. |
| VEGFA NGG site 3 | GGTGAGTGAGTGTGTGCGTGTGG | 92. |
| ZSCAN2 NGG site | GTGCGGCAAGAGCTTCAGCCGGG | 93. |
| FANCF NGG site 1, | GGAATCCCTTCTGCAGCACgTGG | 94. |
| mismatch at 1 | | |
| FANCF NGG site 1, | GGAATCCCTTCTGCAGCAgCTGG | 95. |
| mismatch at 2 | | |
| FANCF NGG site 1, | GGAATCCCTTCTGCAGCtCCTGG | 96. |
| mismatch at 3 | | |
| FANCF NGG site 1, | GGAATCCCTTCTGCAGgACCTGG | 97. |
| mismatch at 4 | | |
| FANCF NGG site 1, | GGAATCCCTTCTGCAcCACCTGG | 98. |
| mismatch at 5 | | |
| FANCF NGG site 1, | GGAATCCCTTCTGCtGCACCTGG | 99. |
| mismatch at 6 | | |
| FANCF NGG site 1, | GGAATCCCTTCTGgAGCACCTGG | 100. |
| mismatch at 7 | | |
| FANCF NGG site 1, | GGAATCCCTTCTcCAGCACCTGG | 101. |
| mismatch at 8 | | |
| FANCF NGG site 1, | GGAATCCCTTCaGCAGCACCTGG | 102. |
| mismatch at 9 | | |
| FANCF NGG site 1, | GGAATCCCTTgTGCAGCACCTGG | 103. |
| mismatch at 10 | | |
| FANCF NGG site 1, | GGAATCCCTaCTGCAGCACCTGG | 104. |
| mismatch at 11 | | |
| FANCF NGG site 1, | GGAATCCCaTCTGCAGCACCTGG | 105. |
| mismatch at 12 | | |
| FANCF NGG site 1, | GGAATCCgTTCTGCAGCACCTGG | 106. |
| mismatch at 13 | | |
| FANCF NGG site 1, | GGAATCgCTTCTGCAGCACCTGG | 107. |
| mismatch at 14 | | |
| FANCF NGG site 1, | GGAATgCCTTCTGCAGCACCTGG | 108. |
| mismatch at 15 | | |
| FANCF NGG site 1, | GGAAaCCCTTCTGCAGCACCTGG | 109. |
| mismatch at 16 | | |
| FANCF NGG site 1, | GGAtTCCCTTCTGCAGCACCTGG | 110. |
| mismatch at 17 | | |
| FANCF NGG site 1, | GGtATCCCTTCTGCAGCACCTGG | 111. |
| mismatch at 18 | | |
| FANCF NGG site 1, | GcAATCCCTTCTGCAGCACCTGG | 112. |
| mismatch at 19 | | |
| FANCF NGG site 1, | cGAATCCCTTCTGCAGCACCTGG | 113. |
| mismatch at 20 | | |
| FANCF NGG site 2, | GCTGCAGAAGGGATTCCATcAGG | 114. |
| mismatch at 1 | | |
| FANCF NGG site 2, | GCTGCAGAAGGGATTCCAaGAGG | 115. |
| mismatch at 2 | | |
| FANCF NGG site 2, | GCTGCAGAAGGGATTCCtTGAGG | 116. |
| mismatch at 3 | | |
| FANCF NGG site 2, | GCTGCAGAAGGGATTCgATGAGG | 117. |
| mismatch at 4 | | |
| FANCF NGG site 2, | GCTGCAGAAGGGATTgCATGAGG | 118. |
| mismatch at 5 | | |
| FANCF NGG site 2, | GCTGCAGAAGGGATaCCATGAGG | 119. |
| mismatch at 6 | | |
| FANCF NGG site 2, | GCTGCAGAAGGGAaTCCATGAGG | 120. |
| mismatch at 7 | | |
| FANCF NGG site 2, | GCTGCAGAAGGGtTTCCATGAGG | 121. |
| mismatch at 8 | | |
| FANCF NGG site 2, | GCTGCAGAAGGcATTCCATGAGG | 122. |
| mismatch at 9 | | |
| FANCF NGG site 2, | GCTGCAGAAGcGATTCCATGAGG | 123. |
| mismatch at 10 | | |
| FANCF NGG site 2, | GCTGCAGAAcGGATTCCATGAGG | 124. |
| mismatch at 11 | | |
| FANCF NGG site 2, | GCTGCAGAtGGGATTCCATGAGG | 125. |
| mismatch at 12 | | |
| FANCF NGG site 2, | GCTGCAGtAGGGATTCCATGAGG | 126. |
| mismatch at 13 | | |
| FANCF NGG site 2, | GCTGCAcAAGGGATTCCATGAGG | 127. |
| mismatch at 14 | | |
| FANCF NGG site 2, | GCTGCtGAAGGGATTCCATGAGG | 128. |
| mismatch at 15 | | |
| FANCF NGG site 2, | GCTGgAGAAGGGATTCCATGAGG | 129. |
| mismatch at 16 | | |
| FANCF NGG site 2, | GCTcCAGAAGGGATTCCATGAGG | 130. |
| mismatch at 17 | | |
| FANCF NGG site 2, | GCaGCAGAAGGGATTCCATGAGG | 131. |
| mismatch at 18 | | |
| FANCF NGG site 2, | GgTGCAGAAGGGATTCCATGAGG | 132. |
| mismatch at 19 | | |
| FANCF NGG site 2, | cCTGCAGAAGGGATTCCATGAGG | 133. |
| mismatch at 20 | | |
| FANCF NGG site 4, | GCTCCAGAGCCGTGCGAATcGGG | 134. |
| mismatch at 1 | | |
| FANCF NGG site 4, | GCTCCAGAGCCGTGCGAAaGGGG | 135. |
| mismatch at 2 | | |
| FANCF NGG site 4, | GCTCCAGAGCCGTGCGAtTGGGG | 136. |
| mismatch at 3 | | |
| FANCF NGG site 4, | GCTCCAGAGCCGTGCGtATGGGG | 137. |
| mismatch at 4 | | |
| FANCF NGG site 4, | GCTCCAGAGCCGTGCcAATGGGG | 138. |
| mismatch at 5 | | |
| FANCF NGG site 4, | GCTCCAGAGCCGTGgGAATGGGG | 139. |
| mismatch at 6 | | |
| FANCF NGG site 4, | GCTCCAGAGCCGTcCGAATGGGG | 140. |
| mismatch at 7 | | |
| FANCF NGG site 4, | GCTCCAGAGCCGaGCGAATGGGG | 141. |
| mismatch at 8 | | |
| FANCF NGG site 4, | GCTCCAGAGCCcTGCGAATGGGG | 142. |
| mismatch at 9 | | |
| FANCF NGG site 4, | GCTCCAGAGCgGTGCGAATGGGG | 143. |
| mismatch at 10 | | |
| FANCF NGG site 4, | GCTCCAGAGgCGTGCGAATGGGG | 144. |
| mismatch at 11 | | |
| FANCF NGG site 4, | GCTCCAGAcCCGTGCGAATGGGG | 145. |
| mismatch at 12 | | |
| FANCF NGG site 4, | GCTCCAGtGCCGTGCGAATGGGG | 146. |
| mismatch at 13 | | |
| FANCF NGG site 4, | GCTCCAcAGCCGTGCGAATGGGG | 147. |
| mismatch at 14 | | |
| FANCF NGG site 4, | GCTCCtGAGCCGTGCGAATGGGG | 148. |
| mismatch at 15 | | |
| FANCF NGG site 4, | GCTCgAGAGCCGTGCGAATGGGG | 149. |
| mismatch at 16 | | |
| FANCF NGG site 4, | GCTgCAGAGCCGTGCGAATGGGG | 150. |
| mismatch at 17 | | |
| FANCF NGG site 4, | GCaCCAGAGCCGTGCGAATGGGG | 151. |
| mismatch at 18 | | |
| FANCF NGG site 4, | GgTCCAGAGCCGTGCGAATGGGG | 152. |
| mismatch at 19 | | |
| FANCF NGG site 4, | cCTCCAGAGCCGTGCGAATGGGG | 153. |
| mismatch at 20 | | |
| FANCF NGG site 6, | GCTTGAGACCGCCAGAAGCaCGG | 154. |
| mismatch at 1 | | |
| FANCF NGG site 6, | GCTTGAGACCGCCAGAAGgTCGG | 155. |
| mismatch at 2 | | |
| FANCF NGG site 6, | GCTTGAGACCGCCAGAAcCTCGG | 156. |
| mismatch at 3 | | |
| FANCF NGG site 6, | GCTTGAGACCGCCAGAtGCTCGG | 157. |
| mismatch at 4 | | |
| FANCF NGG site 6, | GCTTGAGACCGCCAGtAGCTCGG | 158. |
| mismatch at 5 | | |
| FANCF NGG site 6, | GCTTGAGACCGCCAcAAGCTCGG | 159. |
| mismatch at 6 | | |
| FANCF NGG site 6, | GCTTGAGACCGCCtGAAGCTCGG | 160. |
| mismatch at 7 | | |
| FANCF NGG site 6, | GCTTGAGACCGCgAGAAGCTCGG | 161. |
| mismatch at 8 | | |
| FANCF NGG site 6, | GCTTGAGACCGgCAGAAGCTCGG | 162. |
| mismatch at 9 | | |
| FANCF NGG site 6, | GCTTGAGACCcCCAGAAGCTCGG | 163. |
| mismatch at 10 | | |
| FANCF NGG site 6, | GCTTGAGACgGCCAGAAGCTCGG | 164. |
| mismatch at 11 | | |
| FANCF NGG site 6, | GCTTGAGAgCGCCAGAAGCTCGG | 165. |
| mismatch at 12 | | |
| FANCF NGG site 6, | GCTTGAGtCCGCCAGAAGCTCGG | 166. |
| mismatch at 13 | | |
| FANCF NGG site 6, | GCTTGAcACCGCCAGAAGCTCGG | 167. |
| mismatch at 14 | | |
| FANCF NGG site 6, | GCTTGtGACCGCCAGAAGCTCGG | 168. |
| mismatch at 15 | | |
| FANCF NGG site 6, | GCTTcAGACCGCCAGAAGCTCGG | 169. |
| mismatch at 16 | | |
| FANCF NGG site 6, | GCTaGAGACCGCCAGAAGCTCGG | 170. |
| mismatch at 17 | | |
| FANCF NGG site 6, | GCaTGAGACCGCCAGAAGCTCGG | 171. |
| mismatch at 18 | | |
| FANCF NGG site 6, | GgTTGAGACCGCCAGAAGCTCGG | 172. |
| mismatch at 19 | | |
| FANCF NGG site 6, | cCTTGAGACCGCCAGAAGCTCGG | 173. |
| mismatch at 20 | | |

Results and Discussion

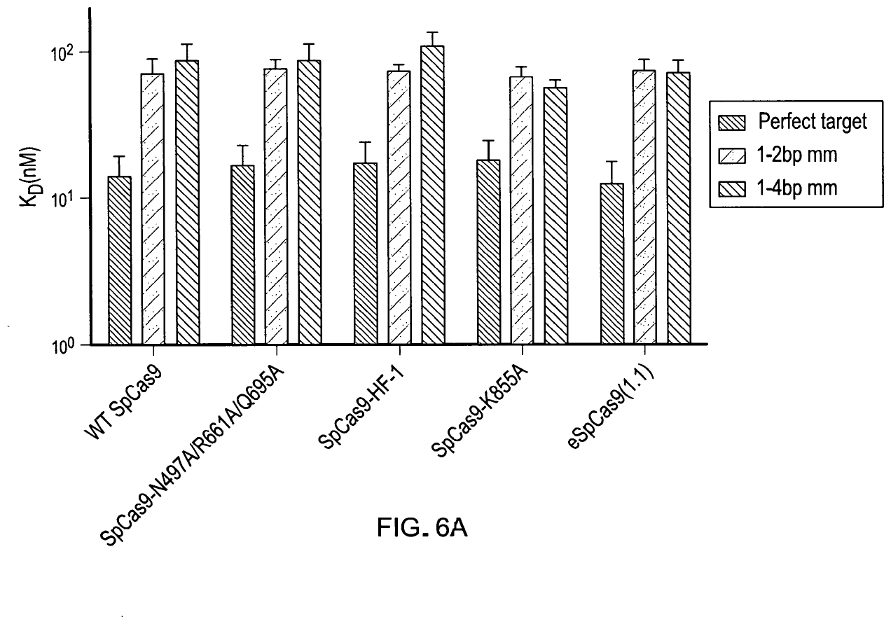

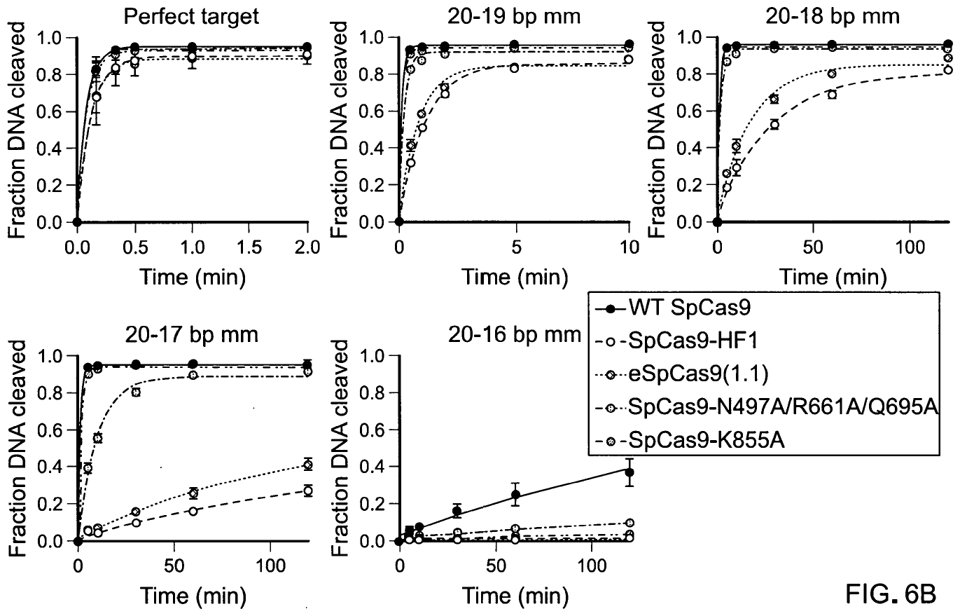

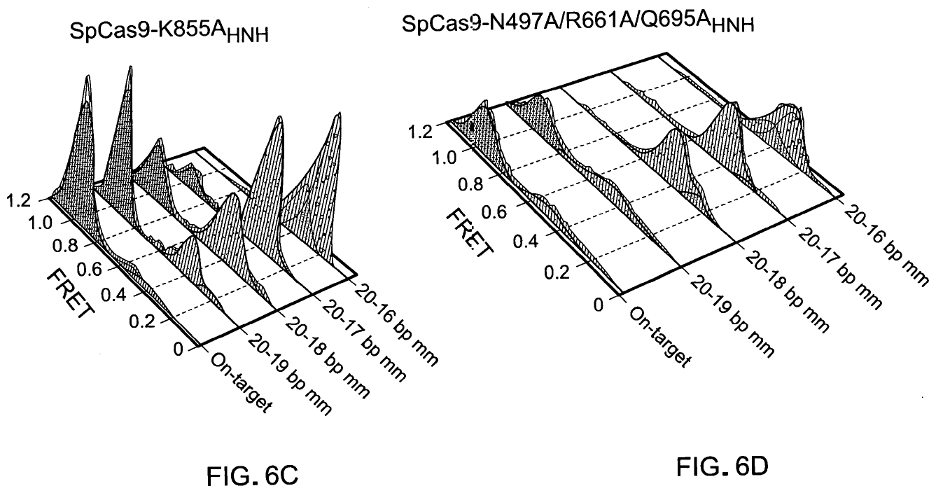

Efforts to abolish off-target activity have motivated the recent development of SpCas9-HF1 and eSpCas9(1.1) variants that contain amino acid substitutions predicted to weaken protein-target DNA interactions^{7,8} (FIG. 1A). To test the hypothesis that SpCas9, when programmed with a single-guide RNA (sgRNA), might contain more energy than is needed for target recognition^{8,11}, we first measured DNA binding affinity and cleavage of SpCas9-HF1 and eSpCas9(1.1) variants in vitro. Contrary to hypotheses that mutating these charged residues to alanine weakens binding, the affinities of these variants for an on-target and PAM-distal mismatched substrates were similar to wild-type (WT) SpCas9 (FIG. 1B, 5A, 6A). While these variants cleave the on-target DNA at similar rates to WT, their cleavage activity was significantly reduced on substrates bearing mismatches (FIG. 6B), suggesting that cleavage specificity is improved through a mechanism distinct from a simple reduction of target affinity.

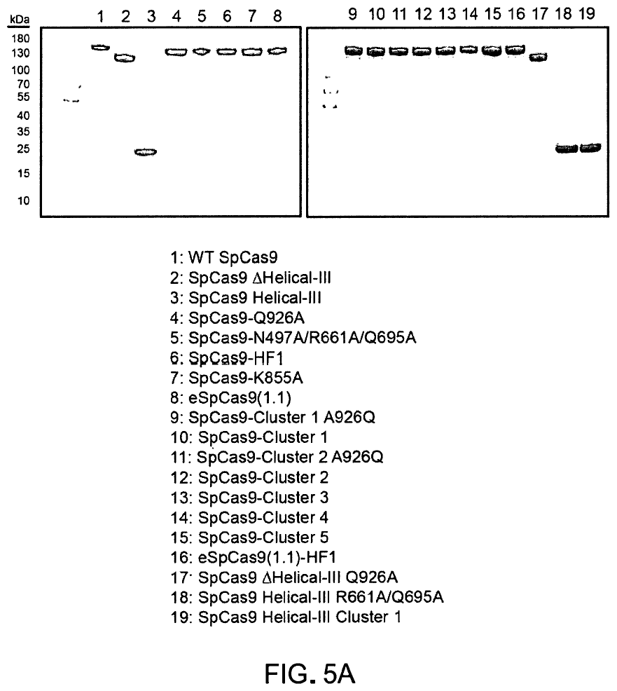

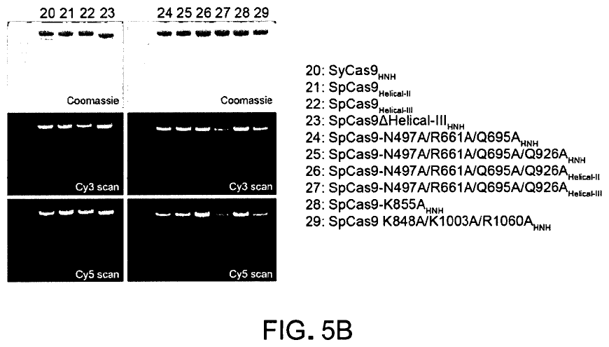

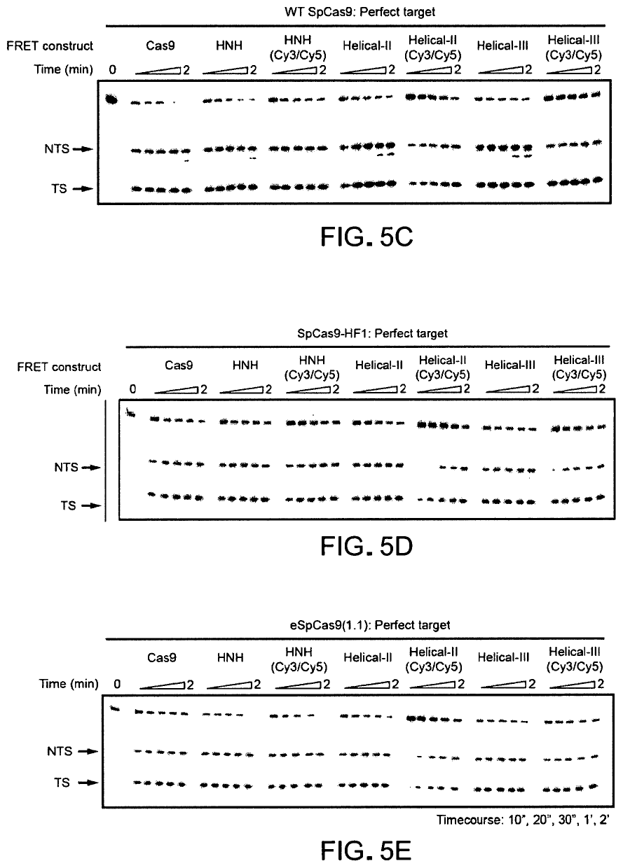

The HNH nuclease in WT SpCas9 undergoes a large conformational change to activate cleavage of both DNA strands¹⁰, but becomes loosely trapped in a catalytically-incompetent intermediate state when bound to mismatched targets⁹ (FIG. 1C-D), suggesting the SpCas9-HF1 and eSpCas9(1.1) variants may employ a more stringent “conformational gating” mechanism to promote off-target discrimination. To test this possibility, we labeled catalytically active, cysteine-light SpCas9 (SpCas9_{HNH}), SpCas9-HF1 (SpCas9-HF1_{HNH}) and eSpCas9(1.1) (eSpCas9(1.1)_{HNH}) with Cy3/Cy5 FRET pairs at positions S355C and S867C to measure HNH conformational states (FIG. 1C, E-F, FIG. 5C-E)¹⁰. Steady-state smFRET histograms revealed that, in ˜30% of SpCas9-HF1_{HNH} molecules, the HNH domain stably occupied the active state with an on-target substrate, while the remaining ˜70% populated the intermediate state. However, when SpCas9-HF1_{HNH} was bound to substrates with PAM-distal mismatches, stable docking of the HNH nuclease was no longer observed (FIG. 1E). This was in contrast to WT SpCas9_{HNH}, eSpCas9(1.1)_{HNH}, and additional high fidelity mutants^{7,8} (SpCas9-N497A/R661A/R695A_{HNH} and SpCas9-K855A_{HNH}), in which the HNH active state was still observed in the presence of mismatches (FIG. 1D, F, 6C-D). We therefore propose that high fidelity variants of Cas9 can reduce off-target cleavage by kinetically trapping the HNH domain in the inactive state when mismatches are present on the DNA substrate.

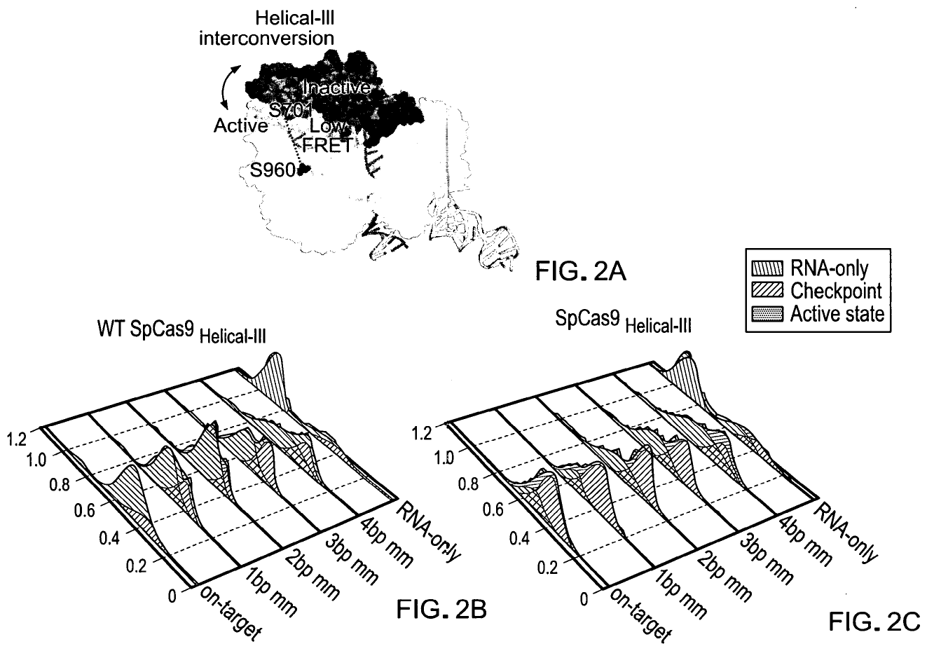

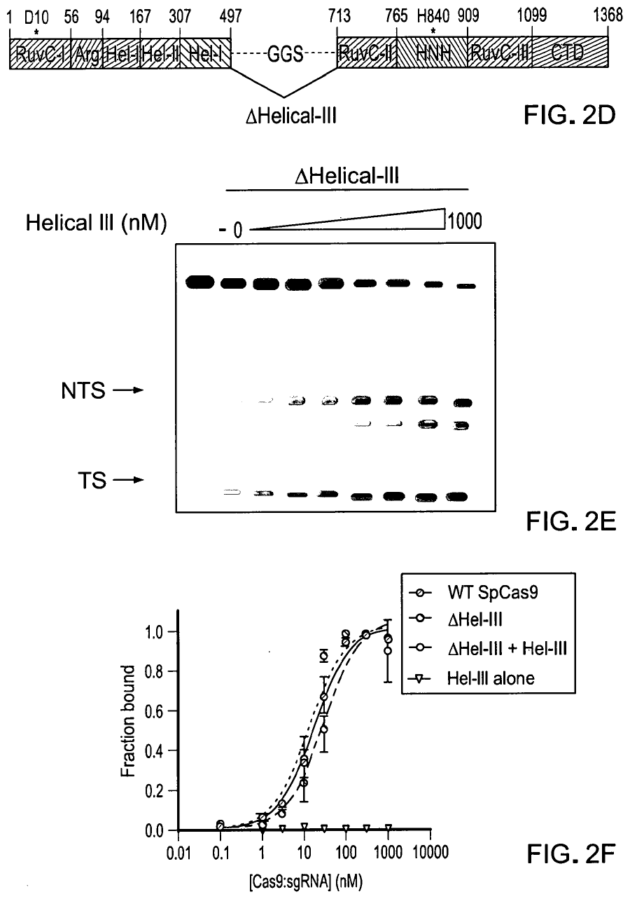

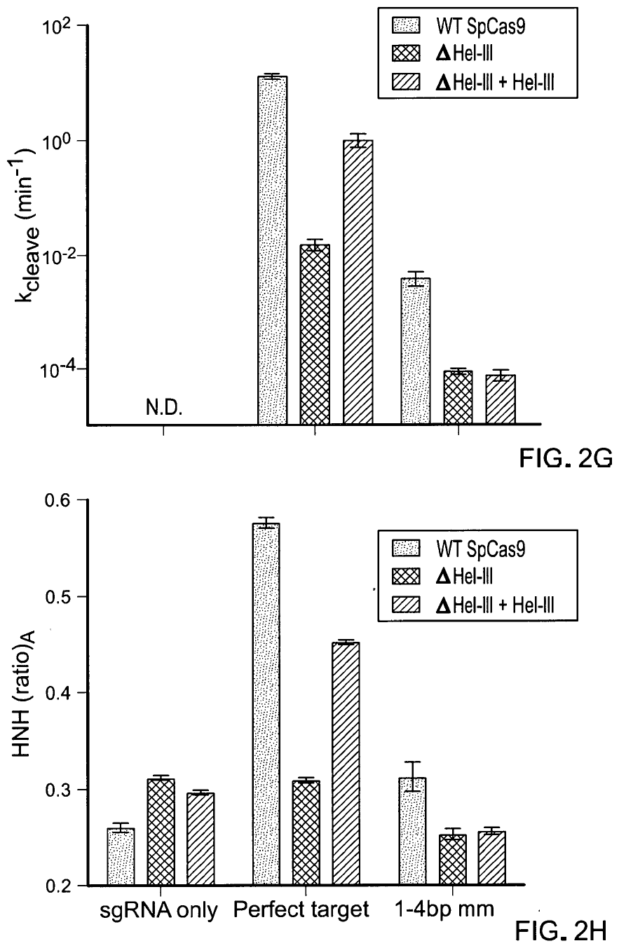

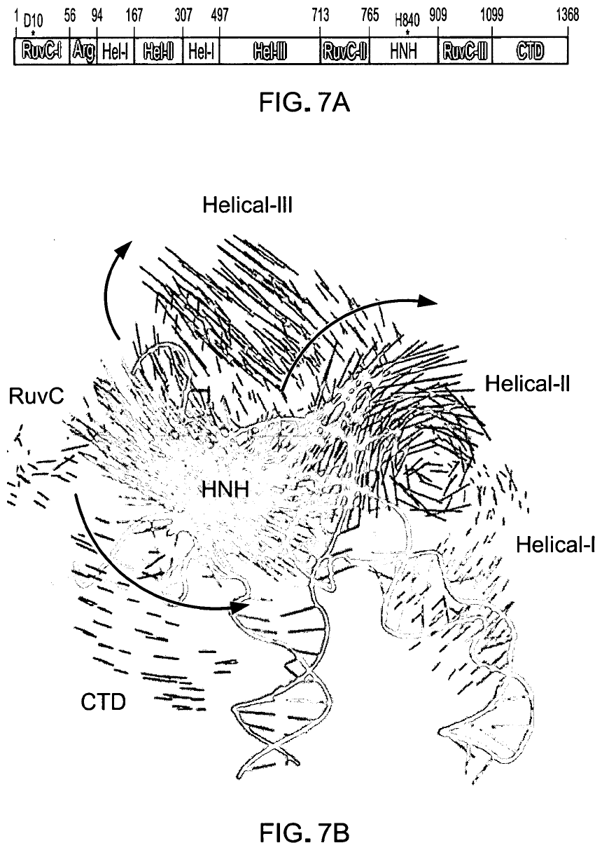

An important question to address is how Cas9 senses RNA/DNA complementarity when the HNH domain does not directly contact nucleic acids at the PAM-distal end. Multiple residues in the Helical-III domain (within the alpha-helical lobe) interact with the RNA/DNA heteroduplex and undergo conformational changes upon target binding¹²⁻¹⁵ (FIG. 7A-B), suggesting that these residues may play a role in sensing complementarity. To test how Helical-III responds to perturbations at the PAM-distal region, we introduced Cy3/Cy5 dyes to cysteine-light SpCas9 at positions S701C and S960C (SpCas9_{Helical-III}) to monitor Helical-III conformational states (FIG. 2A, 5B, C). SpCas9_{Helical-III} primarily occupied a high FRET state (˜0.9) upon sgRNA binding, and switched to an active state (˜0.6) upon binding to a complementary dsDNA target. Unexpectedly, we observed a lower FRET state (˜0.4) in the presence of mismatches, demonstrating that the Helical-III domain shifts further from the RNA-DNA heteroduplex with decreasing nucleic acid complementarity at the PAM-distal end (FIG. 2B). This was distinct from SpCas9-HF1_{Helical-III}, in which the majority of Helical-III occupied the lowest FRET state (˜0.4) with a perfect target substrate (FIG. 2C). These results confirm that Helical-III shifts away from the central channel to accommodate the RNA-DNA heteroduplex¹²⁻¹⁵, but reveal that nucleic acid sensing requires engagement with the Helical-III domain.

To determine whether PAM-distal sensing precedes HNH activation, we deleted Helical-III from WT Cas9 (SpCas9ΔHelical-III) (FIG. 2D). Deletion of Helical-III decreased the on-target cleavage rate by ˜1000-fold compared to WT Cas9 despite retaining near-WT binding affinity with a perfect target, and cleavage was nearly undetectable on a 20-16 bp mismatched substrate (FIG. 2F-G). Unexpectedly, in vitro complementation of SpCas9ΔHelical-III with Helical-III domain in trans rescued the on-target cleavage rate by ˜100-fold in a concentration-dependent manner, but had no effect on cleavage with a PAM-distal mismatched target (FIG. 2E, G). This was further corroborated with bulk FRET experiments, in which supplementing Helical-III in SpCas9ΔHelical-III_{HNH} largely rescued the bulk FRET signal (referred to as HNH (ratio)_{A}) in the presence of a perfect target (FIG. 2G). We propose that Helical-III acts as an allosteric effector to communicate RNA/DNA heteroduplex recognition with HNH nuclease activation.

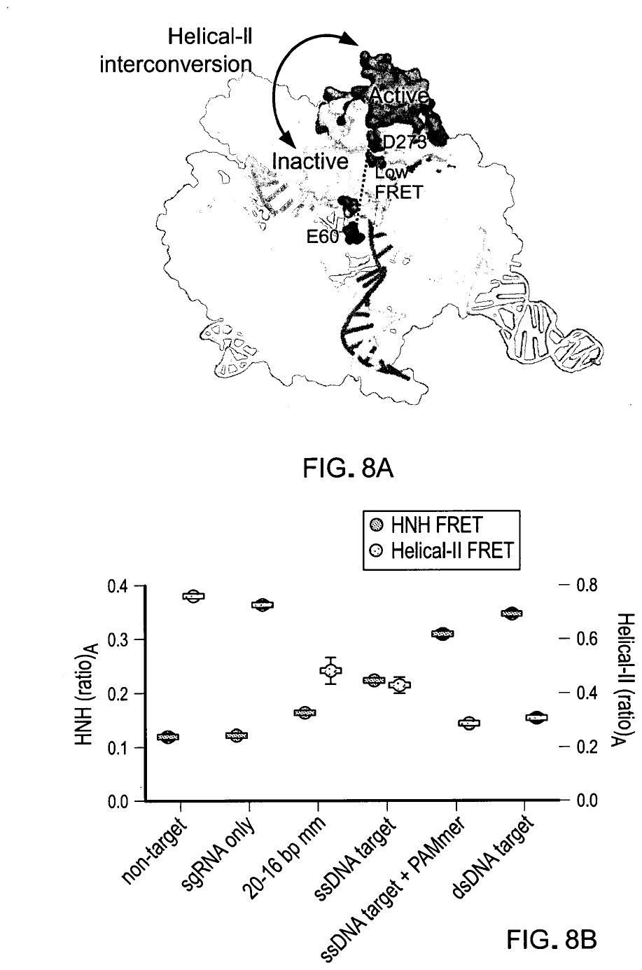

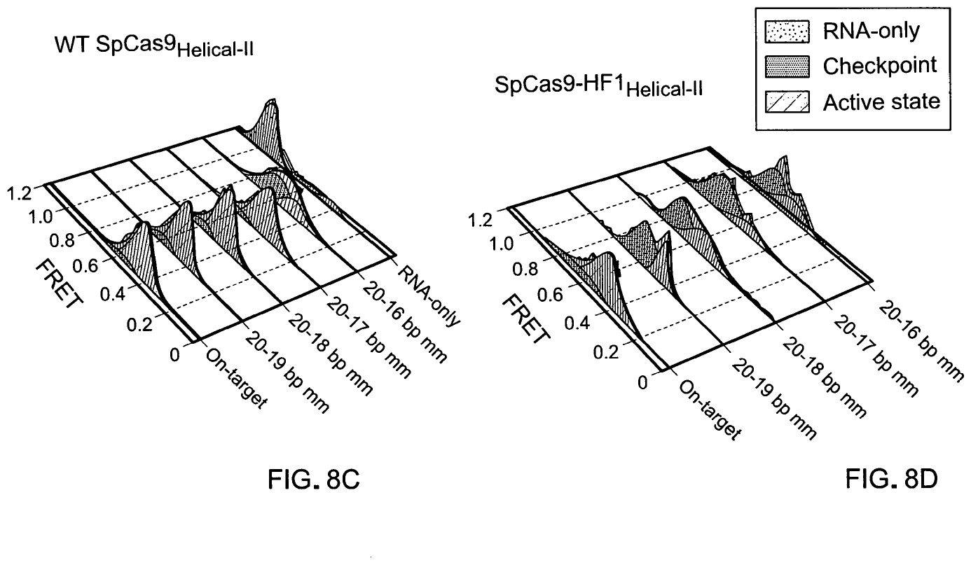

We next asked whether the Helical-II domain facilitated the long-range allosteric interactions that enable coupling of the discontinuous Helical-III and HNH domains. Crystal structures suggested that Helical-II (adjacent to Helical-III) occludes the HNH domain from the active site in the sgRNA-bound state¹⁴, and undergoes a large outward rotation in the dsDNA-bound state¹⁵ (FIG. 7). To test whether the Helical-II domain regulates access of HNH to the target strand scissile phosphate, a cysteine-light SpCas9 was labeled with Cy3/Cy5 dyes at positions E60C and D273C (SpCas9_{Helical-II}) for detecting Helical-II conformational changes (FIG. 5B-C, 8A). We observed reciprocal (ratio)_{A} values between SpCas9_{HNH} and SpCas9_{Helical-II} across multiple DNA substrates in bulk FRET experiments (FIG. 8B), which suggest that the Helical-II and HNH domains undergo coupled movements to ensure catalytic competence. smFRET experiments further confirmed a large opening of Helical-II, from an sgRNA-bound state (˜0.9) to a target-bound state (˜0.5). In contrast to WT SpCas9_{Helical-II}, SpCas9-HF1_{Helical-II} clamps down into an intermediate state (˜0.7) when bound to a target with a single PAM-distal mismatch (FIG. 8C-D). Together with the observation that SpCas9-HF1_{HNH} does not occupy the active state in the presence of PAM-distal mismatches, these experiments demonstrate steric occlusion of the HNH nuclease by Helical-II when bound to off-target substrates.

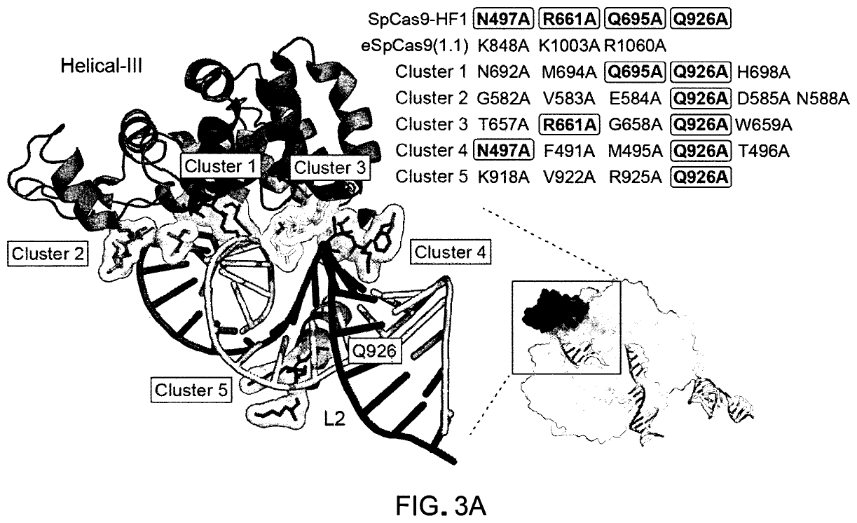

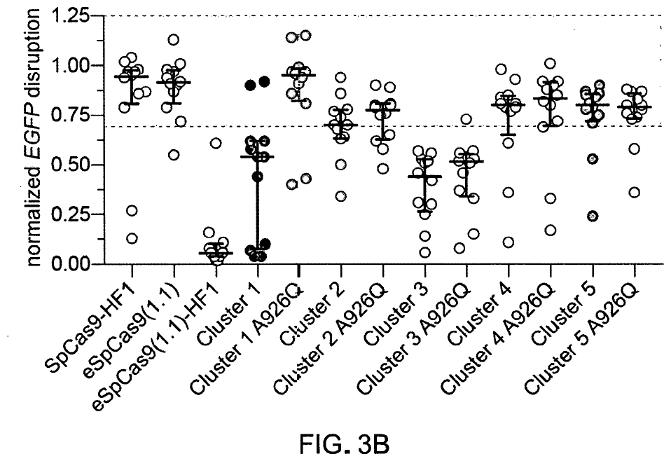

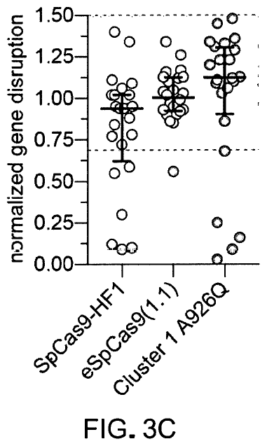

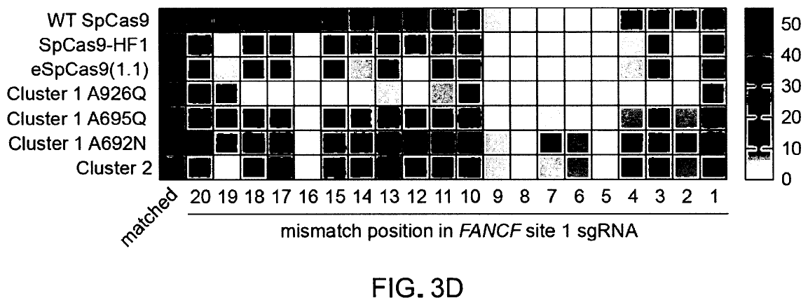

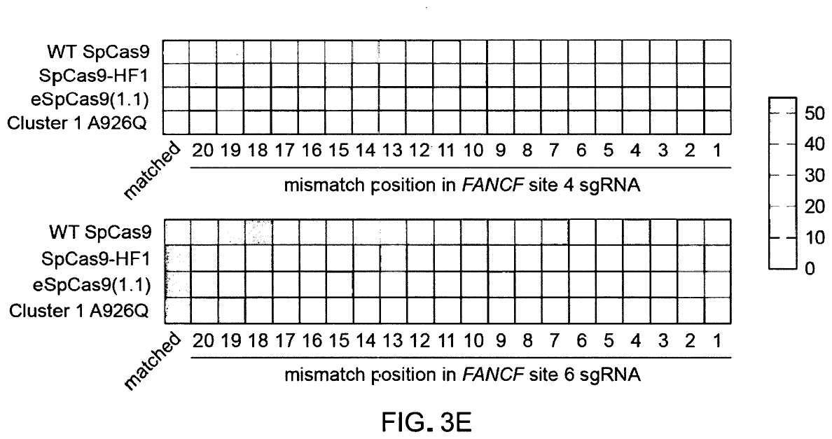

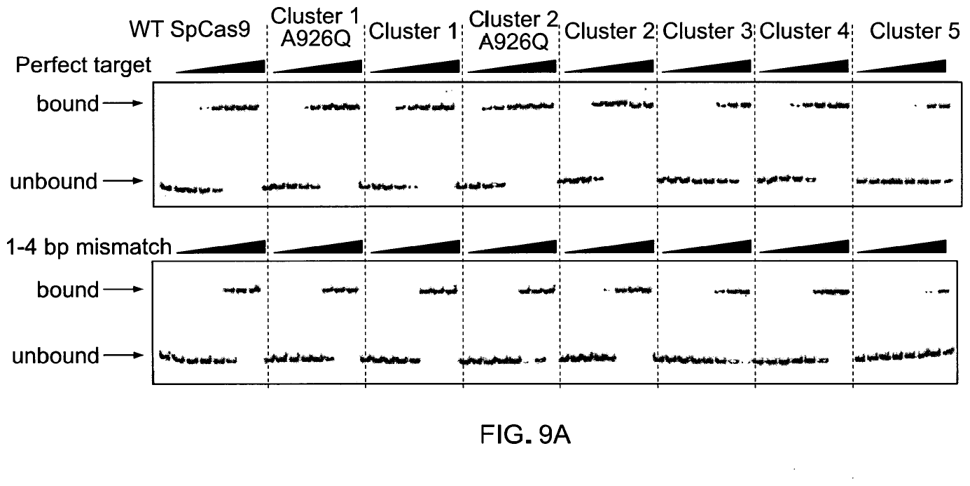

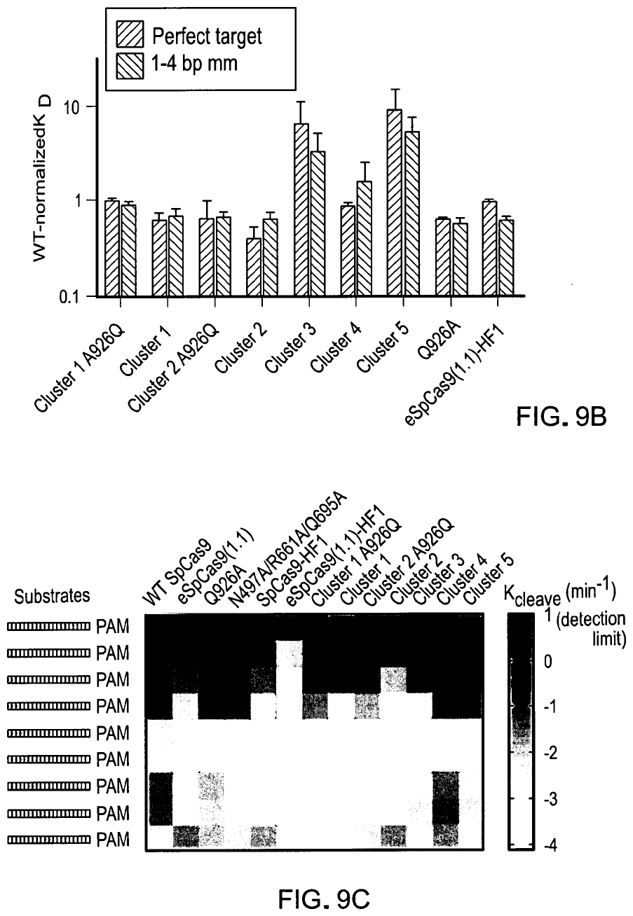

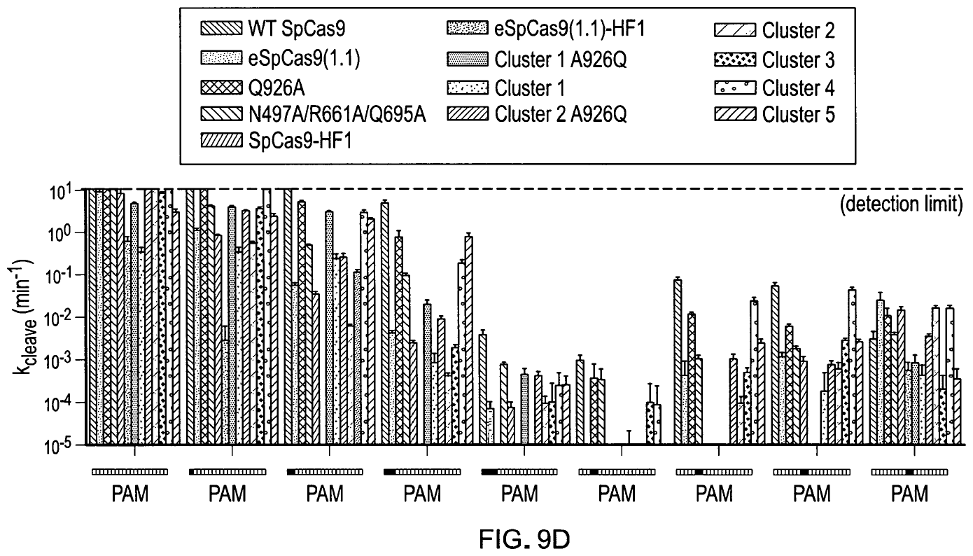

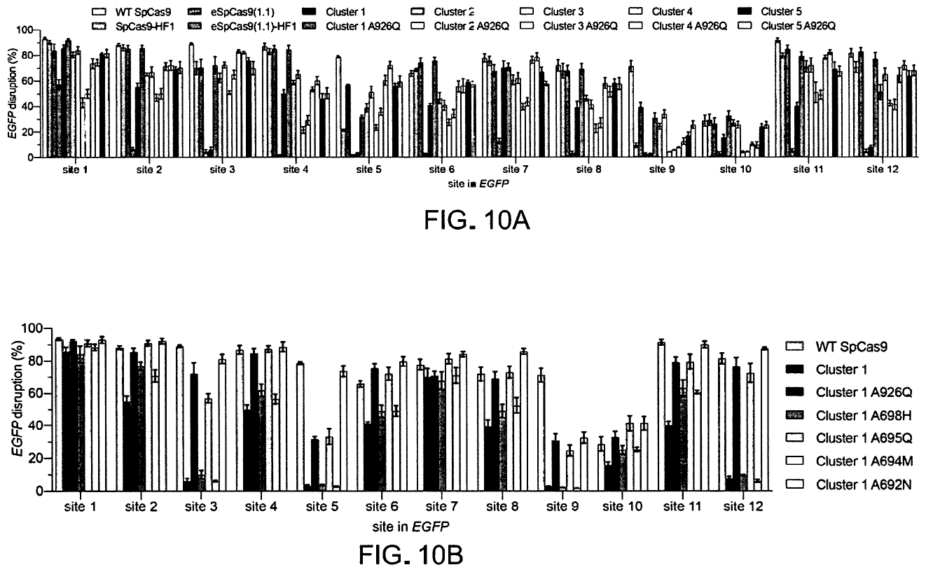

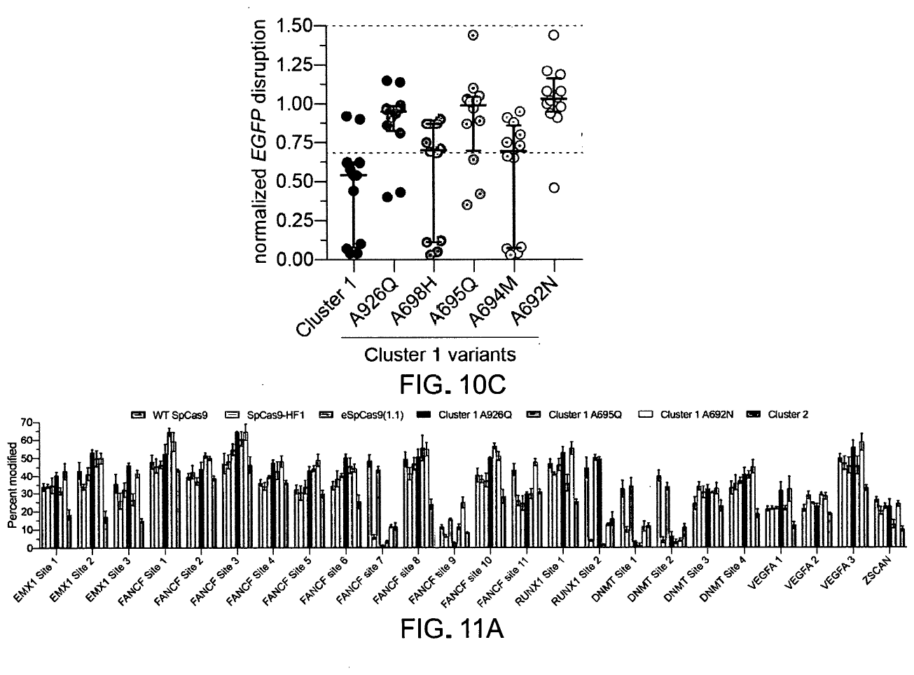

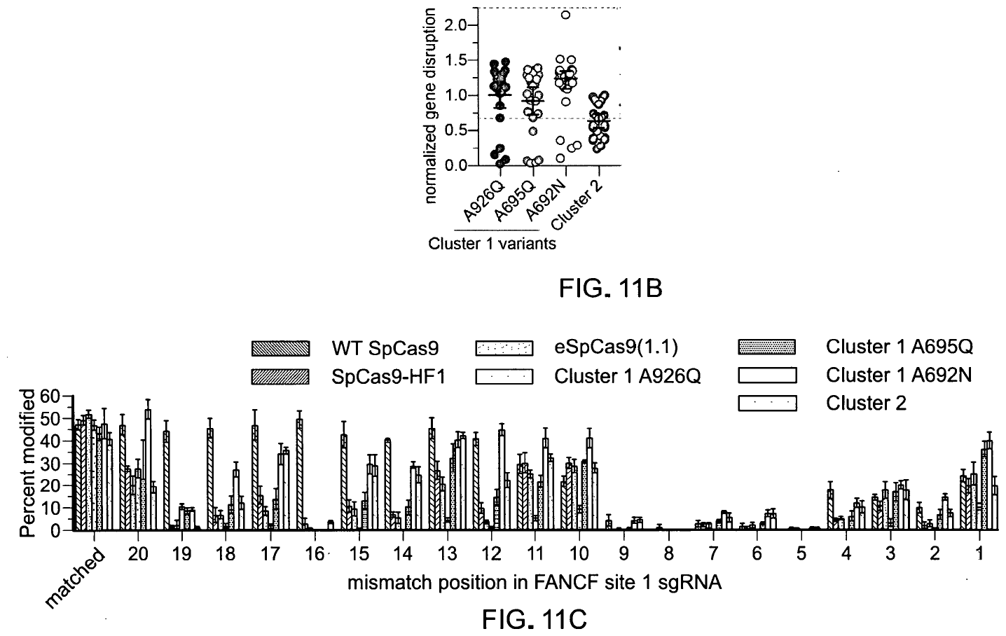

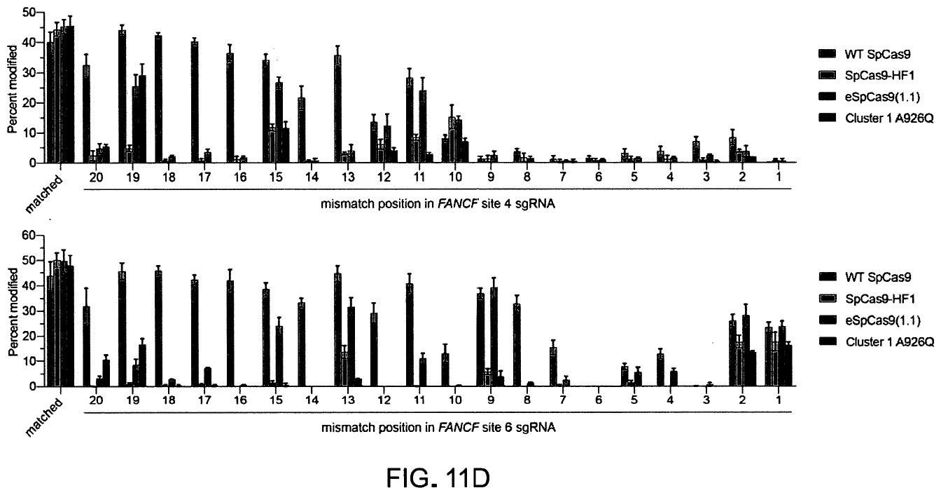

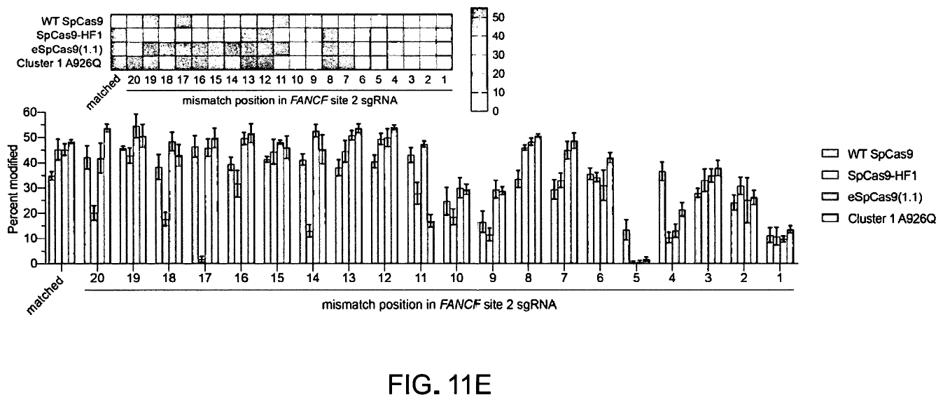

Next, we investigated how this mechanism of conformational trapping could be exploited to design a new suite of hyper-accurate Cas9 variants. We identified five residue clusters, four within the Helical-III domain (Clusters 1-4) and one in the HNH-RuvC linker L2 (Cluster 5) that contact various regions along the RNA/DNA interface. In combination with Q926A, a substitution within L2 that confers specificity⁸, we generated alanine substitutions for each residue within a cluster (FIG. 3A). Equilibrium binding measurements revealed weakened on- and off-target binding with Clusters 3 and 5, while the remaining variants retained affinities comparable to WT SpCas9 (FIG. 9A-B). In vitro cleavage kinetics on a panel of substrates further showed that Clusters 1 and 2 suppressed off-target cleavage, and despite compromised cleavage activity with the Cluster 1 variant, restoring Q926 (A926Q) rescued on-target cleavage kinetics (FIG. 9C-D). We next screened all Cluster variants in human cells using the enhanced GFP (EGFP) disruption assay⁴, and observed on-target activity for Cluster 1 A926Q comparable to SpCas9-HF1 or eSpCas9(1.1), whereas Cluster 2 variants displayed generally lower activity (FIG. 3B, FIG. 10A). Furthermore, Cluster 1 A926Q retained high on-target activity (>70% of WT activity) at 19/24 endogenous sites tested, compared to 18/24 for SpCas9-HF1 and 23/24 for eSpCas9(1.1) (FIG. 3C, FIG. 11A).

We then focused on the specific contributions within Cluster 1 by restoring individual residues to their wild-type amino acids and tested their on-target efficiencies in human cells with the EGFP disruption assay. On-target activity was significantly compromised when N692A/Q695A/Q926A mutations occurred together, but restoring either N692 (Cluster 1 A692N) or Q695 (Cluster 1 A695Q) displayed robust on-target efficiency comparable to Cluster 1 A926Q suggesting differential contributions to activity and potentially specificity (see FIGS. 10B-C and 7A-B for activity against EGFP and endogenous sites, respectively).

Next, to compare the specificities of SpCas9-HF1, eSpCas9(1.1), Cluster 1 A692N, Cluster 1 A695Q, Cluster 1 A926Q, and Cluster 2, we assessed their mismatch intolerance relative to WT using a series of guide RNAs that bear single mismatches against the endogenous human gene target FANCF site 1. Cluster 1 A926Q demonstrated even greater specificity compared to both SpCas9-HF1 and eSpCas9(1.1) in the middle and PAM proximal regions of the spacer (with guide RNAs encoding single mismatches at positions 1 through 18), suggesting that mutating N692A and A695A together may be critical for achieving increased levels of specificity in previously refractory parts of the spacer sequence⁸ (FIG. 3D, 11c). Additional single mismatch tolerance assays on FANCF sites 4 and 6 further corroborated the hyper-accurate specificity of Cluster 1 A926Q against mismatches at positions 1 through 18 (FIG. 3E, 11D). However, all variants failed to substantially improve single mismatch intolerance on FANCF site 2 (FIG. 11E), which may be due to poor sensitivity of the T7E1 assay for the class of insertion or deletion mutations generated at this site.

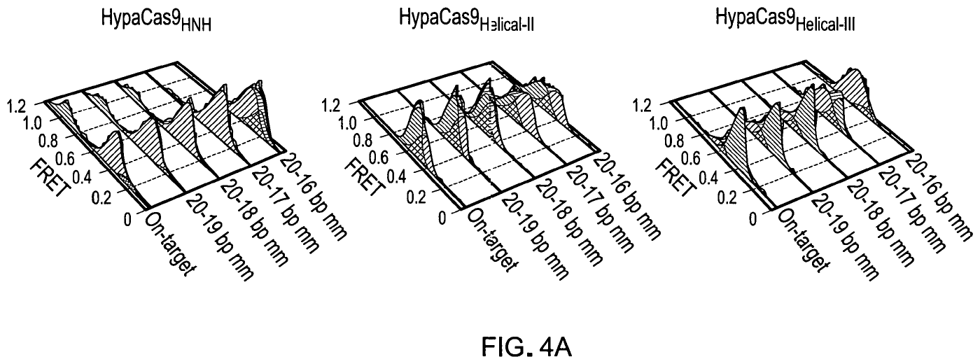

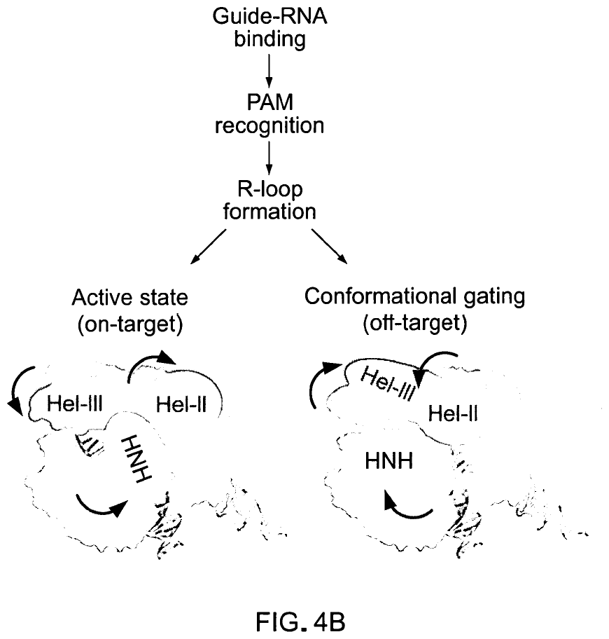

Our finding that Helical-III functions as an allosteric sensor and communicates through Helical-II to activate the HNH domain for cleavage offers a direct explanation for how mismatches within the target sequence trap the HNH nuclease in the conformational checkpoint. Mutating residues that are critical for this interaction, such as in Cluster 1 A926Q or SpCas9-HF1, induces conformational gating by the alpha-helical lobe and prevents HNH activation in the presence of mismatches (FIG. 4). This mechanism is reminiscent of the surveillance complex Cascade from type I CRISPR-Cas systems, in which the R-loop is sensed by the Cse2 “receiver” at the PAM-distal end to trigger R-loop locking and Cas3 nuclease/helicase recruitment¹⁶. Unlike target verification by Cascade, however, SpCas9 lacks an R-loop locking step and has been speculated to rely on protospacer sensing to ensure accurate targeting¹⁷. We demonstrate that protospacer sensing is required for conformational activation and that altering recognition can shift the on- to off-target cleavage differential towards a higher-fidelity Cas9, with minimal disruption to catalytic competence. This outcome may address why nature apparently has not selected for a highly precise Cas9 protein, whose native balance between mismatch tolerance and specificity may be optimized for host immunity. Our study therefore delineates a general strategy for improving Cas9 specificity by tuning conformational activation and offers greater opportunities for designing hyper-accurate Cas9 variants without compromising efficiency.

### REFERENCES

1 Doudna, J. A. & Charpentier, E. Genome editing. The new frontier of genome engineering with CRISPR-Cas9. Science 346, 1258096, doi:10.1126/science.1258096 (2014).
2 Hsu, P. D., Lander, E. S. & Zhang, F. Development and applications of CRISPR-Cas9 for genome engineering. Cell 157, 1262-1278, doi:10.1016/j.cell.2014.05.010 (2014).
3 Mali, P., Esvelt, K. M. & Church, G. M. Cas9 as a versatile tool for engineering biology. Nat Methods 10, 957-963, doi:10.1038/nmeth.2649 (2013).
4 Fu, Y. et al. High-frequency off-target mutagenesis induced by CRISPR-Cas nucleases in human cells. Nat Biotechnol 31, 822-826, doi:10.1038/nbt.2623 (2013).
5 Tsai, S. Q. et al. GUIDE-seq enables genome-wide profiling of off-target cleavage by CRISPR-Cas nucleases. Nat Biotechnol 33, 187-197, doi:10.1038/nbt.3117 (2015).
6 Tsai, S. Q. & Joung, J. K. Defining and improving the genome-wide specificities of CRISPR-Cas9 nucleases. Nat Rev Genet 17, 300-312, doi:10.1038/nrg.2016.28 (2016).
7 Slaymaker, I. M. et al. Rationally engineered Cas9 nucleases with improved specificity. Science 351, 84-88, doi:10.1126/science.aad5227 (2016).
8 Kleinstiver, B. P. et al. High-fidelity CRISPR-Cas9 nucleases with no detectable genome-wide off-target effects. Nature 529, 490-495, doi:10.1038/nature16526 (2016).
9 Dagdas, Y. S., Chen, J. S., Sternberg, S. H., Doudna, J. A. & Yildiz, A. A Conformational Checkpoint Between DNA Binding And Cleavage By CRISPR-Cas9. bioRxiv (2017).
10 Sternberg, S. H., LaFrance, B., Kaplan, M. & Doudna, J. A. Conformational control of DNA target cleavage by CRISPR-Cas9. Nature 527, 110-113, doi:10.1038/nature15544 (2015).
11 Bisaria, N., Jarmoskaite, I. & Herschlag, D. Lessons from Enzyme Kinetics Reveal Specificity Principles for RNA-Guided Nucleases in RNA Interference and CRISPR-Based Genome Editing. Cell Syst 4, 21-29, doi:10.1016/j.cels.2016.12.010 (2017).
12 Nishimasu, H. et al. Crystal structure of Cas9 in complex with guide RNA and target DNA. Cell 156, 935-949, doi:10.1016/j.cell.2014.02.001 (2014).
13 Anders, C., Niewoehner, O., Duerst, A. & Jinek, M. Structural basis of PAM-dependent target DNA recognition by the Cas9 endonuclease. Nature 513, 569-573, doi:10.1038/nature13579 (2014).
14 Jiang, F., Zhou, K., Ma, L., Gressel, S. & Doudna, J. A. STRUCTURAL BIOLOGY. A Cas9-guide RNA complex preorganized for target DNA recognition. Science 348, 1477-1481, doi:10.1126/science.aab1452 (2015).
15 Jiang, F. et al. Structures of a CRISPR-Cas9 R-loop complex primed for DNA cleavage. Science 351, 867-871, doi:10.1126/science.aad8282 (2016).
16 Rutkauskas, M. et al. Directional R-Loop Formation by the CRISPR-Cas Surveillance Complex Cascade Provides Efficient Off-Target Site Rejection. Cell Rep, doi:10.1016/j.celrep.2015.01.067 (2015).
17 Szczelkun, M. D. et al. Direct observation of R-loop formation by single RNA-guided Cas9 and Cascade effector complexes. Proc Natl Acad Sci USA 111, 9798-9803, doi:10.1073/pnas.1402597111 (2014).
18 Jinek, M. et al. Structures of Cas9 endonucleases reveal RNA-mediated conformational activation. Science 343, 1247997, doi:10.1126/science.1247997 (2014).
19 Wright, A. V. et al. Rational design of a split-Cas9 enzyme complex. Proc Natl Acad Sci USA 112, 2984-2989, doi:10.1073/pnas.1501698112 (2015).
20 Kleinstiver, B. P. et al. Engineered CRISPR-Cas9 nucleases with altered PAM specificities. Nature 523, 481-485, doi:10.1038/nature14592 (2015).
21 Reyon, D. et al. FLASH assembly of TALENs for high-throughput genome editing. Nat Biotechnol 30, 460-465, doi:10.1038/nbt.2170 (2012).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An isolated Streptococcus pyogenes Cas9 (SpCas9) protein, with mutations at one, two, three, four, five, six, seven, or more of the following positions: F491, M495, T496, N497, G582, V583, E584, D585, N588, T657, G658, W659, R661, N692, M694, Q695, H698, K918, V922, and/or R925, and optionally at Q926, preferably comprising a sequence that is at least 80% identical to the amino acid sequence of SEQ ID NO:1 with mutations at one, two, three, four, five, six, seven, or more of the following positions: F491, M495, T496, N497, G582, V583, E584, D585, N588, T657, G658, W659, R661, N692, M694, Q695, H698, K918, V922, and/or R925, and optionally at Q926, and optionally one or more of a nuclear localization sequence, cell penetrating peptide sequence, and/or affinity tag.

2. The isolated protein of claim 1, comprising mutations at one, two, three, or all four of the following: N692, M694, Q695, and H698; G582, V583, E584, D585, and N588; T657, G658, W659, and R661; F491, M495, T496, and N497; or K918, V922, R925, and Q926.

3. The isolated protein of claim 1, comprising one, two, three, four, or all of the following mutations: N692A, M694A, Q695A, and H698A; G582A, V583A, E584A, D585A, and N588A; T657A, G658A, W659A, and R661A; F491A, M495A, T496A, and N497A; or K918A, V922A, R925A, and Q926A.

4. The isolated protein of claim 1, comprising mutations: N692A/M694A/Q695A/H698A.

5. The isolated protein of claim 1, comprising mutations: N692A/M694A/Q695A/H698A/Q926A; N692A/M694A/Q695A/Q926A; N692A/M694A/H698A/Q926A; N692A/Q695A/H698A/Q926A; M694A/Q695A/H698A/Q926A; N692A/Q695A/H698A; N692A/M694A/Q695A; N692A/H698A/Q926A; N692A/M694A/Q926A; N692A/M694A/H698A; M694A/Q695A/H698A; M694A/Q695A/Q926A; Q695A/H698A/Q926A; G582A/V583A/E584A/D585A/N588A/Q926A; G582A/V583A/E584A/D585A/N588A; T657A/G658A/W659A/R661A/Q926A; T657A/G658A/W659A/R661A; F491A/M495A/T496A/N497A/Q926A; F491A/M495A/T496A/N497A; K918A/V922A/R925A/Q926A; or 918A/V922A/R925A.

6. The isolated protein of claim 1, further comprising one or more of the following mutations: D1135E; D1135V; G1218R; R1335Q; R1335E; T1337R; D1135V/R1335Q/T1337R (VQR variant); D1135E/R1335Q/T1337R (EQR variant); D1135V/G1218R/R1335Q/T1337R (VRQR variant); or D1135V/G1218R/R1335E/T1337R (VRER variant).

7. The isolated protein of claim 1, further comprising one or more mutations that decrease nuclease activity selected from the group consisting of mutations at D10, E762, D839, H983, or D986; and at H840 or N863.

8. The isolated protein of claim 6, wherein the mutations that decrease nuclease activity are:
(i) D10A or D10N, and
(ii) H840A, H840N, or H840Y.

9. A fusion protein comprising the isolated protein of claims 1-8, preferably comprising one or more mutations that decrease nuclease activity, fused to a heterologous functional domain, with an optional intervening linker, wherein the linker does not interfere with activity of the fusion protein.

10. The fusion protein of claim 9, wherein the heterologous functional domain is a transcriptional activation domain.

11. The fusion protein of claim 10, wherein the transcriptional activation domain is from VP64 or NF-κB p65.

12. The fusion protein of claim 9, wherein the heterologous functional domain is a transcriptional silencer or transcriptional repression domain.

13. The fusion protein of claim 12, wherein the transcriptional repression domain is a Krueppel-associated box (KRAB) domain, ERF repressor domain (ERD), or mSin3A interaction domain (SID).

14. The fusion protein of claim 12, wherein the transcriptional silencer is Heterochromatin Protein 1 (HP1), preferably HP1α or HP1β.

15. The fusion protein of claim 9, wherein the heterologous functional domain is an enzyme that modifies the methylation state of DNA.

16. The fusion protein of claim 15, wherein the enzyme that modifies the methylation state of DNA is a DNA methyltransferase (DNMT) or a TET protein.

17. The fusion protein of claim 16, wherein the TET protein is TET1.

18. The fusion protein of claim 9, wherein the heterologous functional domain is an enzyme that modifies a histone subunit.

19. The fusion protein of claim 9, wherein the enzyme that modifies a histone subunit is a histone acetyltransferase (HAT), histone deacetylase (HDAC), histone methyltransferase (HMT), or histone demethylase.

20. The fusion protein of claim 9, wherein the heterologous functional domain is a biological tether.

21. The fusion protein of claim 20, wherein the biological tether is MS2, Csy4 or lambda N protein.

22. The fusion protein of claim 20, wherein the heterologous functional domain is FokI.

23. The fusion protein of claim 20, wherein the heterologous functional domain comprises a deaminase enzyme, e.g., a cytidine deaminase, and optionally a uracil glycosylase inhibitor (UGI) domain.

24. An isolated nucleic acid encoding the protein of claims 1-8 or the fusion protein of claims 9-23.

25. A vector comprising the isolated nucleic acid of claim 24, optionally operably linked to one or more regulatory domains for expressing the protein of claims 1-8 or the fusion protein of claims 9-23.

26. A host cell, preferably a mammalian host cell, comprising the nucleic acid of claim 24, and optionally expressing the protein of claims 1-8 or the fusion protein of claims 9-23.

27. A method of altering the genome of a cell, the method comprising expressing in the cell or contacting the cell with the isolated protein of claims 1-8 or the fusion protein of claims 9-23, and a guide RNA having a region complementary to a selected portion of the genome of the cell.

28. The method of claim 27, wherein the isolated protein or fusion protein comprises one or more of a nuclear localization sequence, cell penetrating peptide sequence, and/or affinity tag.

29. The method of claim 27, wherein the cell is a stem cell, e.g., an embryonic stem cell, mesenchymal stem cell, or induced pluripotent stem cell; is in a living animal; or is in an embryo.

30. A method of altering a double stranded DNA (dsDNA) molecule, the method comprising contacting the dsDNA molecule with the isolated protein of claims 1-8 or the fusion protein of claims 9-23, and a guide RNA having a region complementary to a selected portion of the dsDNA molecule.

31. The method of claim 30, wherein the dsDNA molecule is in vitro.
